(19) United States
(12) Patent Application Publication
(10) Pub. No.: US 2025/0308650 A1
(43) Pub. Date: Oct. 2, 2025

(54) PERSONALIZED SPINAL IMPLANT AND BIOLOGICS

(71) Applicant: Warsaw Orthopedic, Inc., Warsaw, IN (US)

(72) Inventors: Melissa K. DOBSON, Memphis, TN (US); Jeffrey M. BADURA, Memphis, TN (US); Ian R. DUNKLEY, Memphis, TN (US); Alfonso Santiago FERRNDEZ, Warsaw, IN (US); Todd M. BOYCE, Collierville, TN (US); Daniel A. SHIMKO, Memphis, TN (US); Benjamin T. REVES, Warsaw, IN (US)

(21) Appl. No.: 18/749,302

(22) Filed: Jun. 20, 2024

Related U.S. Application Data
(60) Provisional application No. 63/573,229, filed on Apr. 2, 2024.

(51) Int. Cl. G16H 10/60; G06N 20/00; G06T 17/00; G16H 50/20

(52) U.S. Cl. CPC G16H 10/60; G06N 20/00; G06T 17/00; G16H 50/20

(57) ABSTRACT

Methods and systems are provided for using a machine learning system, such as an artificial neural network, with machine learning or deep learning recognition of clinical data patterns and correlations, with capabilities to integrate generative artificial intelligence, to determine the optimal bone grafting materials and procedures personalized to a patient and/or digital twin of a patient. In an embodiment describe herein, a representation of a location of a bone graft for a patient is accessed. A recommendation including an implant, one or more bone graft materials or biologics for the patient is generated based on applying a representation of the location of the bone graft and health data of the patient within a neural network. The recommendation is displayed at a provider user interface (UI) and/or patient UI.

### CROSS-REFERENCE TO RELATED APPLICATIONS

This application claims priority from U.S. Patent Application No. 63/573,229, filed on Apr. 2, 2024, the contents of which are hereby incorporated by reference herein in their entirety.

### FIELD

The present technology is generally related to technologies for personalizing therapeutic and surgical products and processes, specifically here to personalizing spinal implants, biologics, and bone grafts including clinical decision support using machine learning and/or artificial neural networks.

### BACKGROUND

Bone grafts or bone graft substitutes can be used for a variety of procedures including: repairing bones damaged from trauma (e.g., bone fractures, etc.), assisting bone healing or growth in areas where bone did not previously exist (e.g., intervertebral body spinal fusion, intertransverse process/posterolateral spinal fusion etc.), assisting in bone healing or growth around surgically implanted devices (e.g., spinal fusion, joint replacements, dental implants, etc.), or replacing missing bone for any reason, such as injury, infection or disease. There are a significant number of materials, such as biologics (e.g., natural or bioengineered substances), and combinations of materials, that are available for bone graft procedures to promote bone healing. With the goal of achieving better patient clinical outcomes, a surgeon will typically utilize their prior experience to choose particular materials for a bone graft procedure based on factors such as patient co-morbidities, the size and location of the area to be treated, and the surgeon's preferences. However, the surgeon may be unaware of the characteristics of implants, which may be interbody cages or biologics, and bone graft materials; the surgeon may not be aware of the implant biochemical properties, surface chemistries, and efficacy of all bone graft materials, combinations of bone graft materials with respect to a specific procedure that involves a bone graft and specific health characteristics of the patient. A need exists to better align materials and implants characterized by the technological properties and personalized to the identified patient.

### SUMMARY

The system of the present disclosure recognizes the needs identified above and addresses the patient in an objective data-driven capacity to meet the health needs of both the health provider and the patient in delivering and presenting, respectively, the preferred outcomes. The techniques of this disclosure generally relate to using a machine learning model, such as an artificial neural network (“neural network”), to determine a recommendation of the optimal bone grafting materials and/or procedures for a patient.

In one aspect, the present disclosure provides a computing system. The computing system comprises at least one computer processor, and computer memory having computer-readable instructions embodied thereon, that, when executed by the at least one computer processor, perform operations. The operations comprise receiving patient data for a human patient, the data including a location on the patient for utilizing an implant. The operations further comprise receiving implant characterization data comprising osteoinduction and osteoconduction data characterizing at least one implant type. The operations further comprise determining one or more applicable implants for the patient by applying the patient data and the implant characterization data to an artificial intelligence (AI) model. The operations further comprise generating a recommendation based on the one or more applicable implants. The operations further comprise causing a representation of the recommendation to be presented via a user interface.

In another aspect, the disclosure provides a computer-implemented method. The method includes receiving patient data for a human patient, the data including a location on the patient for utilizing a spinal implant and an osteogenic capacity score for the patient. The method further includes receiving implant characterization data comprising osteoinduction and osteoconduction data characterizing at least one spinal implant type. The method further includes determining one or more applicable implants for the patient by applying at least the patient data and the implant characterization data to an artificial intelligence (AI) model, the AI model trained to output indications of implants that are optimized with regards to osteoconduction, osteoinduction, or osteogenesis of bone graft material of the implants based on prior health data of a patient population identified as optimal clinical outcome. The method further includes generating a recommendation based on the one or more applicable implants. The method further includes causing a representation of the recommendation to be presented via a user interface.

In another aspect, the disclosure provides a surgical guidance system. The surgical guidance system includes a computer processor and is operative to receive, at the computer processor, feedback data, provided by one or more distributed networked computers, the feedback data for a plurality of prior patients who have undergone spinal surgery and characterizing implant-related data for each prior patient. The surgical guidance system is further operative to train an artificial intelligence (AI) model based on the feedback data. The surgical guidance system is further operative to obtain, from at least one of the one or more distributed network computers, pre-operative data characterizing implants of a first patient for surgery. The surgical guidance system is further operative to generate a surgical plan for the first patient based on processing the pre-operative data through the AI model, the surgical implant regarding an applicable implant for the patient. The surgical guidance system is further operative to cause at least a portion of the surgical plan to be presented on a display device.

In another aspect, the disclosure provides a computer-implemented method. The method includes accessing a representation of a location of an interbody or bone graft for a patient. A clinical decision support recommendation, such as bone graft material for the patient, is generated or otherwise determined based on applying a representation of the location of the bone graft and health data of the patient to a data-based model such as a machine learning, model, an artificial intelligence model or neural network, a physics-based model or a combination. The system provides a recommendation to a healthcare provider, such as a surgeon performing the bone graft procedure on the patient. Multivariate approaches may also assimilate cluster analysis, MANOVA, Hotelling's T-squared models, or the like. In the event where the outcome needs to be not only fusion, but also reduced pain or lower infection, for exemplary purposes and not limitation, where more than one outcome matters, then such an approach could be employed. As well, converting any multivariate statistical method to execution by machine learning or in training artificial intelligence is embodied herein.

In another aspect, the disclosure provides that the clinical decision support recommendation is further generated based on applying surgical history data of a surgeon designated to perform the bone graft to the neural network. In another aspect, the disclosure provides the recommendation is further generated based on applying a preferred bone graft material to the neural network. In another aspect, the disclosure provides the recommendation further includes a device of a procedure involving the bone graft. In another aspect, the disclosure provides the recommendation further includes a projected clinical outcome for the bone graft. In another aspect, the disclosure provides the recommendation is displayed via a device of a surgeon designated to perform the bone graft. In another aspect, the disclosure provides the recommendation is displayed via a device of the patient in response to receiving approval of the recommendation. In another aspect, the disclosure provides the bone graft material is prepared via bone graft preparation hardware in response to receiving a selection of the bone graft material. In another aspect, the disclosure provides the bone graft material is prepared via bone graft preparation hardware and automatically loaded onto a robotic system in response to receiving a selection of the bone graft material. In another aspect, the disclosure provides the model is trained to optimize selection of the bone graft material based on osteoconduction, osteoinduction, and osteogenesis, and/or other properties of the bone graft material that maximizes clinical outcome.

In one aspect, the present disclosure provides a non-transitory computer-readable medium storing executable instructions, which when executed by a processing device, cause the processing device to perform operations. The operations include that a representation of a location of a bone graft for a patient is accessed. A recommendation including a device of a procedure involving the bone graft for the patient is generated based on applying a representation of the location of the bone graft and health data of the patient to a neural network. The recommendation is then displayed.

In another aspect, the disclosure provides the recommendation is further generated based on applying surgical history data of a surgeon designated to perform the bone graft to the model. In another aspect, the disclosure provides the recommendation is further generated based on applying a preferred bone graft material to the model. In another aspect, the disclosure provides the recommendation further includes bone graft material for the bone graft procedure. In another aspect, the disclosure provides the recommendation further includes a projected clinical outcome for the bone graft.

In one aspect, the present disclosure provides a computing system with a processor and a non-transitory computer-readable medium having stored thereon instructions that when executed by the processor, cause the processor to perform operations. The operations include that a representation of a location of a bone graft for a patient is accessed. Surgical history data of a surgeon designated to perform the bone graft is accessed. A recommendation comprising bone graft material for the patient is generated based on applying a representation of the location of the bone graft, relative complexity of the planned procedure, health data of the patient, and the surgical history data of the surgeon to a neural network. The bone graft material is prepared via bone graft preparation hardware in response to receiving a selection of the bone graft material by the surgeon.

In another aspect, the disclosure provides the recommendation is further generated based on applying a preferred bone graft material to the model. In another aspect, the disclosure provides the recommendation further includes a device of a procedure involving the bone graft. In another aspect, the disclosure provides the neural network is trained to optimize selection of the bone graft material based on osteoconduction, osteoinduction, osteogenesis and/or other properties of the bone graft material that maximizes clinical outcome and minimizes cost.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 depicts a diagram of an example computing system in which one or more embodiments of the present disclosure can be practiced, in accordance with an embodiment of the present disclosure;

FIG. 2A depicts an example diagram of a model implemented to determine a recommendation of the optimal bone grafting materials and/or procedures for a patient, in accordance with an embodiment of the present disclosure;

FIG. 2B depicts a number of example products and their corresponding osteoinductivity characteristics, in accordance with an embodiment of the disclosure;

FIG. 3A provides an example of a user interface (UI) of a provider computing device for viewing patient data including, according to some embodiments, data that is utilized in a data-based model such as machine learning (ML) or deep learning (DL), a physiology-based model, or a combination of both environments to determine outcomes of optimal implant selection;

FIG. 3B provides an example of a graphical (GUI) on a provider computing device for viewing patient data including enabling a user to select or otherwise access a particular image view and/or interact with the three-dimensional (3D) visualization of the data, and for presenting real-time, objective, data-driven visual recommendations as to the optimal spinal implant, bone grafting materials and/or procedures for a patient, in accordance with an embodiment of the present disclosure;

FIG. 3C provides an illustrative GUI of a provider computing device, for accessing, viewing, and/or selecting patient data, procedural protocol(s), materials, implants, and other surgical/medical preferences in accordance with an embodiment of the present disclosure;

FIG. 3D provides an example of a GUI of a provider computing device for viewing a recommendation of the spinal implant, optimal bone grafting materials, tools/instruments, and/or procedures for a patient, in accordance with an embodiment of the present disclosure;

FIG. 3E provides an example of a GUI for viewing a surgical decision support UI, including a recommendation of the suggested implants, bone graft materials, bone graft substitutes, biologics, tools, and/or procedures for a patient, in accordance with an embodiment of the present disclosure;

FIG. 4A depicts an exemplary embodiment as to a programmatic creation of a calibrated virtual patient (i.e., a digital patient twin);

FIG. 4B depicts an example process flow for programmatically generating a patient treatment therapy profile using simulated bone grafting on virtual patients, in accordance with an embodiment of the disclosure;

FIG. 5 is a flow diagram showing a method for determining a recommendation of the optimal bone grafting materials and/or procedures for a patient, in accordance with an embodiment of the present disclosure;

FIG. 6 is a depiction of a block diagram of a computing environment suitable for use in implementing aspects of the technology described herein;

FIG. 7 is a block diagram of an example computing environment suitable for use in implementing aspects of the technology described herein; and

FIG. 8 is a representative decision support system integrated to utilize ML/DL and generative AI to display real-time visualizations of data that correlate a patient with a suggested surgical implant, biologic, tools and/or procedure, in accordance with embodiments herein; and

FIG. 9 provides an example of a GUI configured for a patient and for viewing aspects of a recommendation of the suggested implants, bone graft materials, biologics, tools, and/or procedures for a patient, in accordance with an embodiment of the present disclosure.

### DETAILED DESCRIPTION

### Definitions

Various terms are used throughout the description of embodiments provided herein. A brief overview of such terms and phrases is provided here for ease of understanding, but more details of these terms and phrases is provided throughout.

A “bone graft” refers to bone tissue or synthetic materials to replace or to repair bone structures, whether bones damaged from trauma (e.g., bone fractures, etc.), assisting in bone healing or growth in areas where bone didn't previously exist (e.g. intervertebral body spinal fusion, intertransverse process/posterolateral spinal fusion, etc.), assisting in bone healing or growth around surgically implanted devices (e.g., joint replacements, dental implants, etc.), or replace missing bone for any reason, such as injury, infection or disease. Bone grafts can be transplanted from the patient's own body (e.g., an autograft), from a donor (e.g., an allograft, such as demineralized bone matrix (DBM), cellular bone matrix (CBM), viable bone matrix (VBM), etc.), or from a synthetic bone graft (e.g., using materials that can support bone growth, such as ceramic, plastic, composites, etc.). Bone graft substitutes shall be included in the definition of bone grafts herein. Biologics is also inclusive of bone graft and bone graft substitutes.

The term “biologics” is used broadly herein to refer to natural or bioengineered substances used to promote bone healing and regeneration. Biologics includes bone grafts (e.g., autograft bone, synthetics, or allograft). Without limitation, examples of biologics include, bone morphogenetic proteins (BMPs) and other bone growth factors, small molecules, peptides, platelet-rich plasma (PRP), stem cells, DBM, bone marrow aspirate (BMA), collagen and other natural scaffolds, CBM, VBM, synthetic grafts utilizing materials such as hydroxyapatite, tricalcium phosphate, and bioglass, and/or the like. Biologics can be derived from various sources, including the patient's own body (e.g., autologous), a donor, or produced synthetically. Biologics can be incorporated into bone graft materials to enhance bone healing and integration. In some examples, liquid or gel forms of bone graft materials, such DBM, BMA, etc., can be injected via a syringe. In some examples, robotic systems, such as robotic arm systems, can be used to assist surgeons in planning and/or performing surgeries, such as by providing precise positioning of surgical instruments, implants, bone grafts, etc. via real-time 3-dimensional imaging. Biologics may also comprise any use of the examples above, individually or in combination.

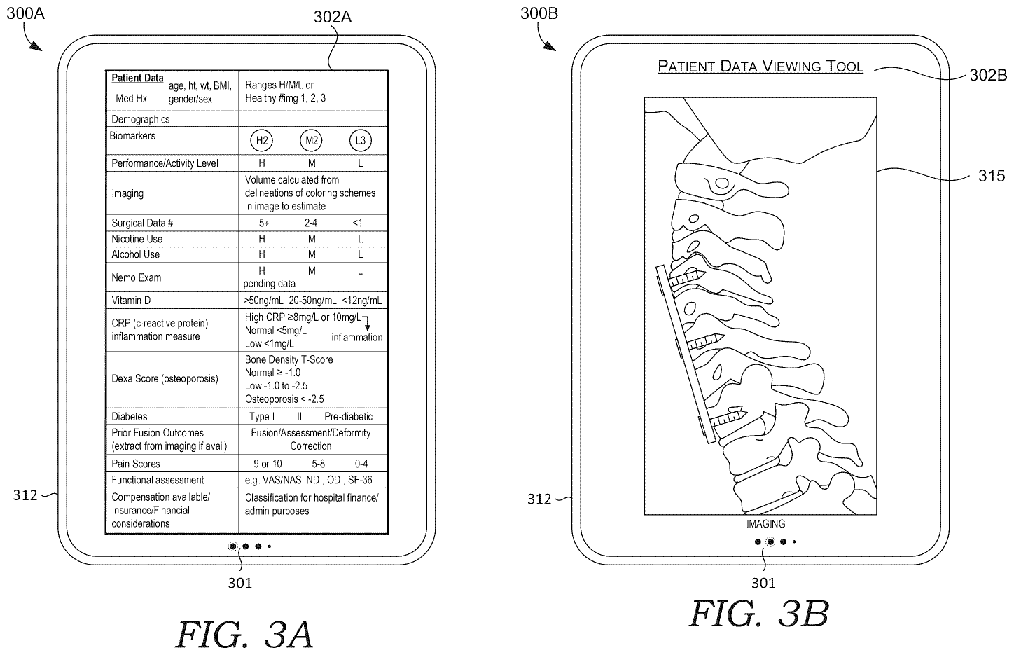

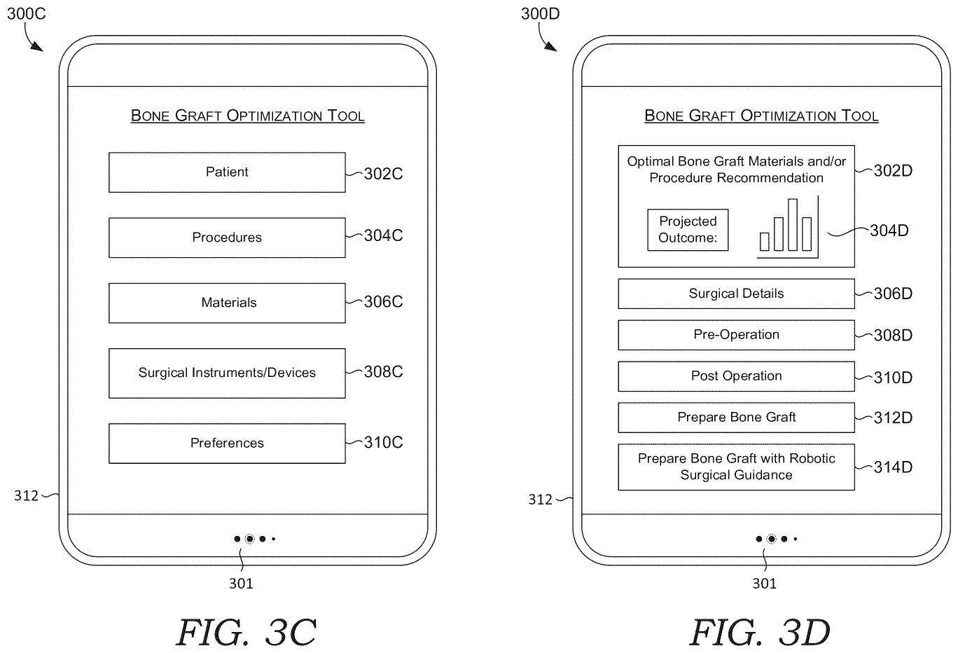

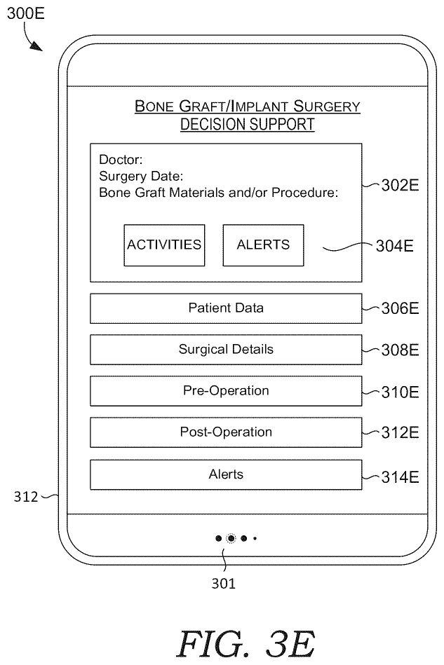

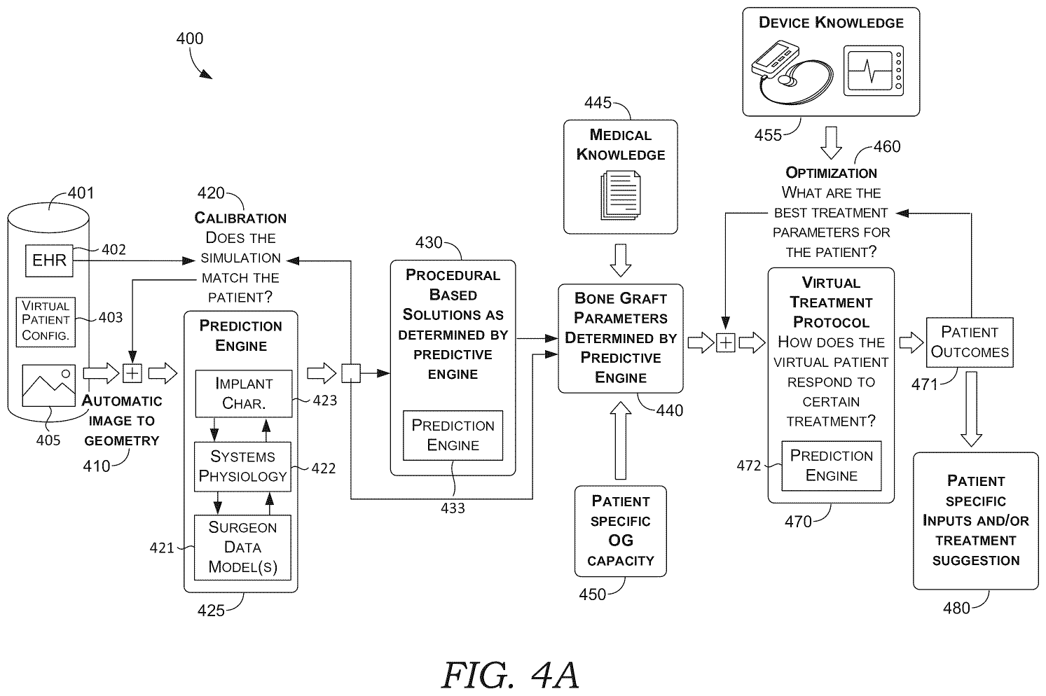

“Biomarker” (short for biological marker) are a biological measure of a biological state, specifically, a characteristic that is objectively measured and evaluated as an indicator of normal biological processes, pathogenic processes or pharmacological responses to a therapeutic intervention. Biomarkers are the measures used to perform a clinical assessment such as blood pressure, common blood panel including cholesterol levels, protein levels, vitamin deficiencies, sugar levels, nicotine usage, or other diagnostic procedures (e.g., DEXA scan (as used in assessment of osteopenia/osteoporosis), etc.) and are used to monitor and predict health states in individuals or across populations so that appropriate therapeutic intervention can be planned. For exemplary purposes, and not limitation, Vitamin D is a biomarker. A number of biomarkers are indicated in the user interface of FIG. 3A.

“Osteogenic factors” or “Bone growth factors” refer to substances that are naturally occurring or added to certain bone grafts and biologics to enhance the healing process; osteogenic factors or genes encoding these factors have been injected directly into fractures or osteotomies to accelerate regeneration, based on the assumption that the factors will initiate the entire cascade of regenerative events. Examples of bone growth factors include, BMPs (e.g., BMP-2), transforming growth factor-beta (TGF-β), fibroblast growth factors (FGFs), platelet-derived growth factor (PDGF), and insulin-like growth factors (IGFs). Bone growth factors can be derived from various sources, including the patient's own body (e.g., autologous), a donor, or produced synthetically. Bone growth factors can be incorporated into bone graft materials to enhance bone healing and integration.

Generally, successful bone healing involves three mechanisms of action (MOA): osteogenesis, osteoconduction, and osteoinduction. Although all three MOAs are necessary for a successful bone grafting procedure, not every bone graft will be required to contain all three MOAs. Generally, the patient is the best source of osteogenic cells, with osteoconduction serving as a scaffold and osteoinduction as the stimulator for growth.

“Osteogenesis” refers to living cells, such as osteoblasts, that form new bone. The success of any bone grafting procedure is dependent on having enough bone forming (e.g., osteogenic) cells in the area. For example, iliac crest bone graft (ICBG) (e.g., a type of autograft) includes more mesenchymal stem cells (MSCs) than local bone. Local bone (e.g., an autograft from the surgical site) is often made up of more cortical bone than cancellous bone and includes fewer MSCs. However, the presence of MSCs does not make a bone graft osteogenic on its own as the MSCs require a signal, such as BMP, to differentiate into osteoblasts.

“Osteoconduction” (OC) refers to the ability of materials to serve as a scaffold (e.g., a three-dimensional structure that provides a framework or support for the growth of new tissue) onto which bone cells can attach, migrate, grow, and divide. In this way, the bone healing response is conducted through the graft site, similar to a vine using a trellis for support. Osteogenic cells generally work much better when they have a matrix or scaffold for attachment. For example, DBMs containing bone fibers produce a greater osteoconductive structure than particles. Ceramics are strictly osteoconductive scaffolds and fall in the category of autograft extender or bone void filler.

“Osteoinduction” (OI) refers to the capacity of growth factors in the body to attract, proliferate, and differentiate MSCs or immature bone cells into osteoblasts to form healthy bone tissue. Generally, most of these signals (e.g., biological signals or cues that promote bone formation and healing) are part of a group of protein molecules called bone morphogenetic proteins, or BMPs, and are found in normal bone. Highly osteoinductive bone grafts have been evaluated as an autograft alternative in certain indications. Osteoinductive grafts induce the active recruitment and stimulation of stem cells that differentiate into osteoblasts and form bone.

An “Osteogenic (OG) Capacity Score” is a rating scale which takes into account the patient, surgical site, and procedure to determine how a particular type of bone grafting material or biologic will deliver the intended result and desired outcome.

“Intradiscal” refers to anything located within the intervertebral disc space of the spine. In the context of spine surgery, “interbody” refers to the implant device inserted in the space between two adjacent vertebrae in the spine. This space is normally occupied by an intervertebral disc, which provides cushioning and allows for movement and flexibility in the spine. In spinal surgery, interbody techniques are often used to address various spinal disorders by replacing a damaged or degenerated disc with an interbody cage or fusion device with bone graft or biologics in the interbody space. This process is known as interbody fusion. These procedures typically involve the use of an interbody device, such as a cage, which is inserted into the empty disc space. This cage can be made from various materials like titanium, carbon fiber, or a bio-compatible polymer, and it often contains bone graft material to promote fusion between the vertebrae. For exemplary purposes, and not limitation, intradiscal surgery refers to the placement of vertebral implants/interbodies or reconstruction of vertebrae within lumbar, thoracic, and cervical portions of the spine. In addition, intradiscal procedures may also include:
Annulo-nucleoplasty (The Disc-FX procedure)
Cervical intradiscal radiofrequency lesioning
Coblation percutaneous disc decompression
Disc arthoplasty (e.g. mechanical devices used in cervical and lumbar to replace the intervertebral disc)
Intradiscal biacuplasty (IDB)/intervertebral disc biacuplasty/cooled radiofrequency
Intradiscal electrothermal annuloplasty (IEA)
Intradiscal electrothermal therapy (IDET)
Intradiscal thermal annuloplasty (IDTA)
Nucleoplasty (also known as percutaneous radiofrequency thermomodulation or percutaneous plasma discectomy)
Percutaneous (or plasma) disc decompression (PDD) or discectomy
Percutaneous intradiscal radiofrequency thermocoagulation (PIRFT)/intradiscal radiofrequency thermomodulation/percutaneous radiofrequency thermomodulation
Radiofrequency annuloplasty (RA)
Targeted disc decompression (TDD) Intradiscal injections (not an all-inclusive list) (e.g., methylene blue, hyaluronate, ozone, oxygen/ozone, bone marrow concentrate, chymopapain, platelet rich plasma (PRP), mesenchymal stem cells, glucocorticoids, hyaluronidase, growth factors, etc.) In some embodiment, bone grafts or biologics are utilized in the above intradiscal procedures. In other embodiments, bone grafts or biologics are not utilized in the above intradiscal procedures.

“Extradiscal” refers to anything located outside the intervertebral discs in the spine. The term is often used in the context of spinal surgery or spinal conditions. In the human spine, the intervertebral discs are the soft, cushion-like structures situated between the vertebrae, and they help in providing flexibility and absorbing shock. Extradiscal structures or regions would include: vertebrae, facet joints, spinal canal, nerve roots, ligaments, muscles, and/or the like. For example, extradiscal pathologies might include facet joint arthritis, spinal stenosis, vertebral fractures, or issues with spinal ligaments or muscles. Extradiscal procedures would be those targeting these areas, rather than the intervertebral discs.

“Chemotaxis” refers to the movement of an organism or cell in response to a chemical stimulus. Cells exhibiting chemotaxis will move toward or away from specific chemicals in various biological contexts such as immune response, wound healing, and development.

The term “implants,” as used herein, refers to medical devices such as interbodies, intradiscal devices, biologics, bone graft materials, bone growth factors or other similar material intended to be implanted in the body to achieve its therapeutic effect.

Model inputs means the type of data that the model receives. These data types are given their ordinary meaning, so separate definitions are not provided here. Statistical Machine Learning is the type of machine learning that is closest to conventional programming. In this learning mode, the AI/ML system develops statistical models and draws inferences from them. As more training data is provided, the system adjusts the statistical model and improves its ability to analyze or make predictions. Deep Learning is an ML model type “that incorporates neural networks in successive layers in order to learn from data in an iterative manner.” Supervised Learning means that the input data has been labeled or categorized to assist the AI/ML system process it. This pre-processing may be manual, automated, or some combination. Unsupervised Learning means that the training data provided to the AI/ML system has not been categorized or labeled. It is most often used with very large data sets where categorization would be impractical-training an email spam filtering system, for example. Semi-Supervised Learning simply means that the AI/ML system trains on a mixture of categorized and uncategorized data. Reinforcement Learning is a different type of machine learning. In reinforcement learning, the AI/ML system does not learn from a training data set; rather it learns a task by trial and error. A robot learning to climb a set of stairs or navigate over obstacles is one example of reinforcement learning. A locked model has its algorithm fixed upon release. This algorithm is only changed or updated when the system is retrained. A continuously updating model keeps learning and modifying its algorithm even when processing new (not training) data. Output refers both to the type of information that the AI/ML system generates and to the purpose or use of that output. Content means that the AI/ML system generates usable media of any type, such as a plain-language summary of medical records for a patient, a video showing selected excerpts of a medical procedure, or extracts from ECGs showing arrhythmic episodes, or generative content, which is predicted content that may be generated in response to an input prompt. Classifications are an output form that divides the input data into one or more groups, such as categorizing polyps as noncancerous, precancerous, or cancerous, and categorizing ECGs as sinus rhythm or atrial fibrillation. Predictions are forecasts about future events, possibly with associated probabilities, such as the likelihood of developing a particular disease or condition given a patient's past and current state. Recommendations, such as clinical decision support recommendations, are suggestions to a human actor (such as a patient or health care provider), which the human may accept or reject. In contrast to recommendations, an AI/ML system can decide and act autonomously, such as choosing and delivering a dose of medicine, or modifying the setup parameters in a medical device.

In another aspect, numerous publications which have studied the impacts of various patient factors or bone graft selection on patient fusion outcomes and patient clinical outcomes may be utilized in the model.

### Overview

Implants during spine surgery may include interbody cages, rods, screws, bone graft or bone graft substitutes, biologics, or any combination. The bone graft, bone graft substitute (whether autograft to synthetic material), or other biologic is utilized to replace or to repair bone structures, whether bones damaged from trauma (e.g., bone fractures, etc.), assisting in bone healing or growth in areas where bone didn't previously exist (e.g. intervertebral body spinal fusion, intertransverse process/posterolateral spinal fusion, etc.), assisting in bone healing or growth around surgically implanted devices (e.g., joint replacements, dental implants, etc.), or replace missing bone for any reason, such as injury, infection or disease. Bone grafts can be transplanted from the patient's own body (e.g., an autograft), from a donor (e.g., an allograft, such as demineralized bone matrix (DBM), cellular bone matrix (CBM), viable bone matrix (VBM), etc.), or from a synthetic bone graft (e.g., using materials that can support bone growth, such as ceramic, plastic, composites, etc.). In addition, approaches for use of implants or biologics and bone graft could be utilized in other therapy or surgical procedure. For example, and not limitation, the modeling to personalize an implant (e.g. interbody cage, rod/screws, joint replacement, bone graft or biologic, or wound care options) with a specific patient as based on the patient demographics, geography, surgical/therapeutic site, histology, pathology, and other patient-specific characteristics. In knee surgery, for exemplary purposes, the model could be beneficial in selecting the best therapy to treat knee pain due to osteoarthritis. Some osteochondral grafts are scaffolds, while others include cells and growth factors. In addition to osteogenic capacity, chondrogenic capacity would also be incorporated into the model.

In another aspect, the personalized model determines the surgical approach as an open procedure, or minimally invasive such as with navigation approaches. In such instances, surgery may not be the preferred treatment and instead and injectable steroid or pain therapeutic. Other modifying factors may be integrated as need to better address the patient and directed physician needs.

With the goal of achieving optimal patient clinical outcomes, a surgeon will typically utilize their prior experience to choose particular materials for a bone graft procedure based on factors such as patient co-morbidities, the size and location of the area to be treated, the relative complexity of the planned surgical procedure, and the surgeon's preferences. A surgeon will utilize their experience to choose the materials for the bone graft procedure based on factors, such as the size and location of the area to be treated, the patient's condition, the relative complexity of the planned procedure, and the surgeon's preferences. However, the surgeon may not be aware of the efficacy of all bone graft materials and combinations of bone graft materials with respect to a specific procedure that involves a bone graft and specific health characteristics of the patient.

Currently, the surgeon selects an implant, particularly a biologic for discussion here, based on a variety of considerations including, but not limited to, the patient, surgical site, surgical procedure, prior use of the product by that particular surgeon, familiarity with the performance of the product, and similarity of patients who have been impacted by the use of a particular product. Such selections of bone graft material may also be based upon suggestion in the literature, medical and/or scientific preferences of peers, or even designated by a sales representative as based on prior use or prior historical preferences of a surgeon in the use of a product. Accordingly, use of implants and selection criteria have utilized a surgeon's knowledge of a patient's medical records, diagnosis, and health interpretation in view of likely physical characteristics of the bone grafting material, such as the hydration component or malleability.

Embodiments of the present disclosure are directed to the automated, programmatic determination of the recommended implant, optimal bone grafting materials and/or procedures in an efficient and effective manner. In this regard, data regarding the patient and the provider (e.g., the surgeon) can be efficiently and effectively utilized by a trained machine learning system, which comprises one or more artificial neural networks or other machine learning models, trained on data regarding bone graft materials and procedures to determine the implants, tools, optimal bone grafting materials, procedures, and/or recovery therapies. Generally, embodiments described herein comprise a computing system on which operates a machine learning system, such as a neural network, to determine a recommendation for the implants, suggested bone grafting materials and/or procedures for a patient, particularly in reference to bone graft materials. In this regard, the computing system operating the trained machine learning system provides a clinical decision support platform, or a solution recommendation to leave the decision making to the surgeon. For example, a trained neural network system comprises an objective outcome driven platform trained to determine a recommendation of an implant, alone or in combination with tools/instruments, and suggestions of optimal bone graft material selections and/or procedure(s). In some implementations, for instance, the implant suggestion comprises a bone graft material with or without the use of an interbody for use in a particular spinal procedure. Some embodiments of the outcome driven platform identifies optimal bone graft characteristics, e.g., osteoconduction, osteoinduction, and osteogenesis, physical attributes of the bone graft product, compression resistance of the bone graft, malleability, hydration capacity, hydration component, surface chemistry, and/or nanostructures within the bone graft material of the implanted construct to maximize clinical outcome as it correlates personalized data of a patient, diagnosis and/or treatment plan for a particular patient and/or as it configures to minimize cost based on product cost and administrative inventory processes. Additionally, space maintenance of the implant may be incorporated. The correlated data can also be integrated within the platform to minimize cost as based on a) minimizing volume of bone graft material needed, b) identifying the appropriate type of implant for bone graft material and optimize procedure success and reduce revision procedures, and c) use of historical data, costs associated with procedure, inventory, and reimbursement rates. In this regard, a user, such as a surgeon, may select, via user interface of a computing application, an indication of a procedure that involves a particular implant and/or a bone graft for a patient. Administrative decisions at the hospital level or through insurance are also integrated. For example, the cost of revision surgery or other related future healthcare expenses, including outcomes from those procedures may be integrated.

Continuing this example implementation, the trained machine learning system is configured to utilize various aspects of data, which can include, without limitation, the patient's electronic health records (“EHRs”), also known as electronic medical records (EMRs) or other data regarding the patient; historical surgical data regarding the surgeon designated to perform the bone graft procedure; other healthcare provider data, such hospital-related or insurance claims-related data, and/or data regarding the surgeon's preferences or selections of aspects of a procedure, to determine the recommendation of the optimal bone grafting material and/or procedure for the patient. The recommendation of the optimal bone grafting material and/or procedure for the patient is output to the device of the user for display and/or stored. In one implementation, the provider can utilize the recommendation to manually prepare the bone grafting material via manual manipulation or via a device, such as a syringe, or for insertion of the bone graft into the localized region of a patient during the procedure. In another implementation, the provider can select the recommendation in the application in order to cause bone graft material preparation hardware in communication with the application to automatically prepare the bone grafting material.

In operation, as described herein, a healthcare provider (e.g., a surgeon, doctor, nurse, counselor, health insurance administrator, and/or medical specialist, such as a medical materials or device manufacturer, supplier, sales representative, or medical consultant for bone graft materials, tools, or aspects thereof) selects a representation of a procedure involving a bone graft for a patient in a user interface of an application. For example, a surgeon selects a location of a spinal disc removal for a patient that would utilize a bone graft. In some embodiments, the surgeon selects the various characteristics of the procedure, such as the type of implants (e.g., a titanium, carbon fiber, or a bio-compatible polymer cage, fixation devices, and any other device implanted during a surgical surgery that utilizes a bone graft), the type of bone graft materials available for the procedure (e.g., whether ICBG (Iliac Crest Bone Graft is available for the patient, access to specific autografts, allografts, or synthetic bone grafts), instruments available for the procedure (e.g., such as whether the surgeon has access to a robotic system for assisting with the bone graft procedure) and/or any preferences of the surgeon (e.g., such as particular grafting materials that the surgeon identifies).

A machine learning system, such as a neural network, is trained to determine a recommendation of a bone graft material and/or procedure that optimizes the bone graft characteristics, including e.g., osteoconduction, osteoinduction, and osteogenesis, physical characteristics, surface chemistry, biocompatibility, among others, of the implanted construct, as correlated with the demographics, histology, pathology, imaging, and/or diagnosis, or treatment options of a patient to optimized preferred and identified clinical outcomes. Note that other data-based models, including physics-based models or a combination of both may be integrated. Such outcomes may reflect fusion rates, reduced pain, maximize return to work times, reduce hospital stay, and/or minimize costs and/or revision procedures. Historical data regarding bone graft procedures is represented in the machine learning system (e.g., a neural network) to correlate with the digitized information and data retrieved from a particular patient and provide an optimal bone graft selection to facilitate decision making by the surgeon, other health provider, nursing staff, operating room (OR) staff, or sales representative. The trained machine learning system utilizes, for example, patient-related data, such as the patient's EHRs, historical surgical data regarding the surgeon's use of a particular bone graft product designated to perform the bone graft procedure, and/or data regarding the user's selections to determine the recommendation of the optimal implant, optimal bone grafting material and/or procedure for the patient. In some embodiments, the trained machine learning system determines a recommendation of the optimal bone grafting material, along with recommendations regarding the optimal procedure. As such, the system may integrate with a digital smart implant or interbody, with another computational modeling system for spine surgery or robotics, imaging analytics, including any systems of hardware and/or software integrated with a data information hub for use with robotic procedures and/or automation. For example, the machine learning system can recommend an optimal implant, such as a structural allograft interbody spacer, or a titanium, carbon fiber, or a bio-compatible polymer cage for an interbody fusion or recommend an extradiscal fixation device, screw or otherwise for the procedure along with the optimal bone grafting materials suggested for the bone graft portion of the procedure.

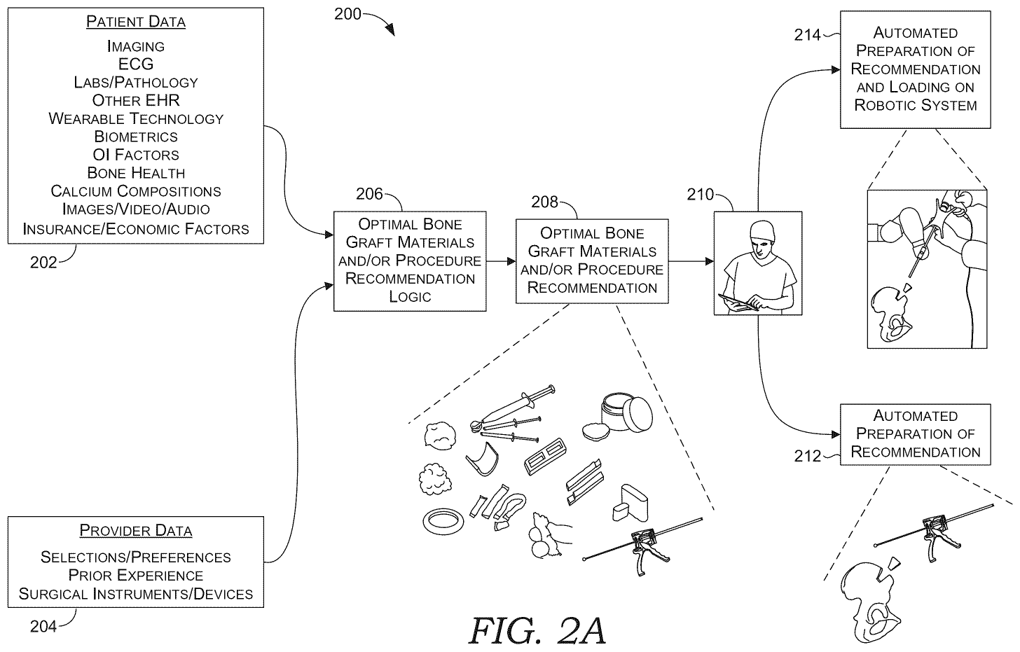

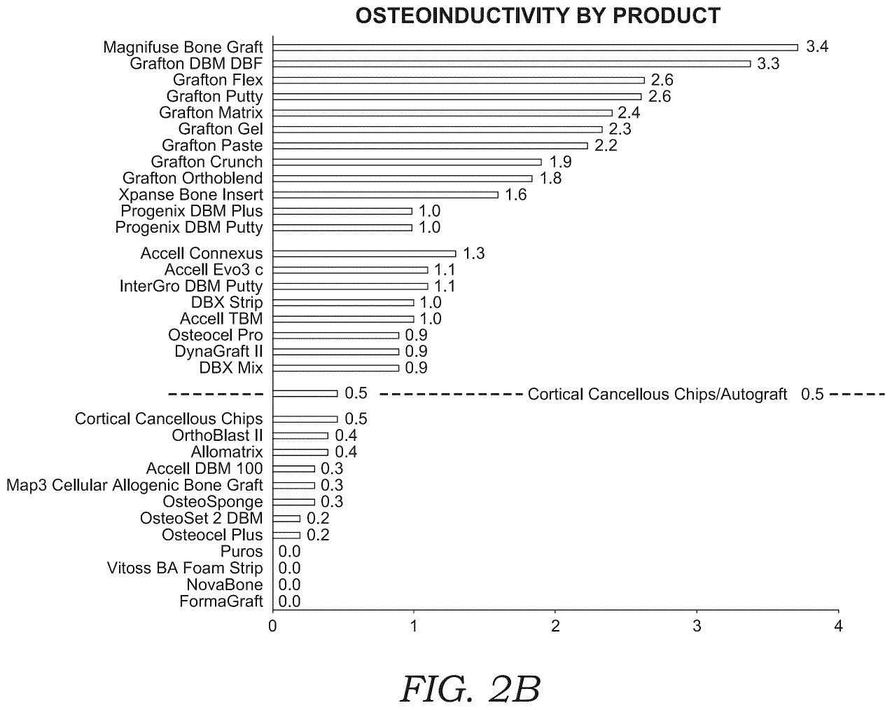

In some embodiments, the machine learning model is configured to utilize osteoconductivity characteristics of various therapeutic and surgical products. For example, FIG. 2B depicts a number of example products and their corresponding osteoconductivity characteristics, any of which may be used included in some embodiments the machine learning model.

The patient-related data, such as the patient's EHRs, include various data regarding the patient, such as but not limited to age, weight, height, nutritional status, and other personal demographic data and/or biometric data. For example, the patient's EHRs may include bone health data, blood work, blood pressure, pain scales or other related detail, and health monitoring information, imaging for the patient, such as thermal imaging, computed tomography (CT) scans, magnetic resonance imaging (MRI) scans, X-RAYs, positron emission tomography (PET) scan, ultrasound, DEXA scans, any variation and/or a combination. Generally, the patient's EHRs can include any health data for the patient, such as an electrocardiogram (ECG or EKG), labs/pathology, wearable technology data, such as that from sensors, biometrics, osteogenesis imperfecta (OGI), or “brittle bone disease” (i.e. diagnosis of a genetic or heritable disease in which bones fracture (break) easily, often with no obvious cause or minimal injury. OI is also known as brittle bone disease, and the symptoms can range from mild with only a few fractures to severe with many medical complications), bone health scores, calcium compositions, vitamin deficiencies, images/video/audio of the patient, data from genomics tools, such as a third party database, diagnostic records, blood testing, histology, blood pressure, heart rate, recurring status, compliance with medicines and prescribed treatment, activities, fusion rates, pain thresholds and assessments, economic status, insurance coverage and/or reimbursements rates or payouts diagnostics, social conditions, psychological conditions, physical or mental health conditions or stress conditions, religious preferences that might impact bone graft preference, geographical location, bone health, blood work, blood pressure, pain, and health monitoring information, and any other information regarding the patient may incorporated into the patient record. The EHR may also include data related to the immediate pathology being treated. This might include descriptions of the spinal deformity, relative angulation and/or slippage of the vertebral bodies, the planned vertebral levels to be treated, prior operations at those levels and if successful, infection status, presence of existing hardware, presence of prior fusions, presence of prior osteotomies, etc.

In this regard, a computing system operating the machine learning system, comprises a personalized treatment system and computational modeling system that determines one or more recommendation(s) of the implant individually or in combination with optimal bone grafting material, tools and/or procedure for the identified patient based on health data for the patient such that a decision support user interface presents the optimal materials for the particular patient and surgeon preference to achieve the clinical outcome as desired. Economic factors of the patient (e.g., insurance coverage) can also be integrated, including reimbursement rates for providers, among other cost considerations as based on selected product, procedure cost, etc. as corresponding to the medical provider selections suggested by the system at the user interface to minimize the costs, if so desired in the selection of the bone grafting materials and/or procedures.

Healthcare provider data, such as the data regarding a surgeon expected to perform a procedure or treatment on the patient, can include, without limitation, any data regarding the particular surgeon, other surgeons that may be involved in the procedure or treatment, a hospital, organization, or facility associated with the patient or procedure (e.g. data from a particular hospital system, an insurance company, the Veterans Administration, etc.). For example, data regarding the surgeon can include any historical preferences of the surgeon, prior experience of the surgeon, such as the outcomes of prior surgical procedures (e.g., procedures involving bone grafts, procedures similar to the procedure selected for the patient, and/or etc.), time to perform the prior procedures, data regarding learning experiences regarding surgical procedures, and/or the like. In some embodiments, the data regarding the surgeon may include data regarding a group of surgeons (e.g., a group at a particular hospital or in a particular healthcare network). In this regard, the computing system operating the trained machine learning system can determine the recommendation of the optimal bone grafting material and/or procedure for the patient based on prior experience of the surgeon performing the surgery.

The data regarding the user's selections can include data regarding the user's preferences as selected by the user via an application. For example, a surgeon may select specific preferences for specific materials and/or procedures. As another example, a medical specialist (such as a medical materials or device manufacturer, supplier, sales representative, or medical consultant for bone graft materials, tools, or aspects thereof) selects a specific preference for a particular product or group of products, such as a particular allograft or demineralized bone matrix (DBM) product, cellular or viable bone matrix (CBM or VBM, respectively) product, or synthetic graft. Cellular/viable bone matrices have an increased number of viable cells from a donor material (allograft) to promote bone growth. Synthetic graft may likely be preferred in younger patients who have healthier and/or stronger bones, as well as to minimize risks associated with transplanting allograft tissue of a donor into a healthy young individual.

Allograft and DBM products can have effective osteoinductive and osteoconductive capabilities as indicated across a range of scoring. For example, the osteoinductive potential of bone grafts can be measured in a standard rat model. A variety of products, such as those indicated above (and others) can be categorized in a scoring system based on characterization of product in combination with the use of product with specific patient presenting diagnosis, disease, but also overall patient health. Such scoring may be indicative of a product's osteoinductive potential, the scoring of which add previously unrealized opportunity to enhance and personalize bone graft selection as based on patient data and correlated with bone growth characteristics.

Other attributes of a product may be correlated to lyophilized product that utilizes hydration component(s), injectable materials that are hydrated in a syringe or injectable device/gun, putty-type malleable materials, ranges of textured bone graft including small to larger size chips within the putty, and product that can be hydrated and dehydrated, mixed with autograft, or packaged in mesh, cannula, or other containment system. In some embodiments, the preferences can be selected for a single user, such as a single surgeon, or a group of users, such as a group of surgeons at a particular hospital or in a particular healthcare network. In some embodiments, the preferences are crowdsourced from many users, such as by presenting recommended preferences selected by most surgeons. In some embodiments, the application may provide prior selections, such as previous settings/configurations, and can include with links to information about the previous cases so that the user can access the information regarding the previous cases.

In this regard, the computing system operating the trained machine learning system comprises a clinical decision support platform that is configured to determine a recommendation of an optimal bone grafting material and/or procedure for the patient based on preferences of the user, such as preferences of the surgeon, or as based on successful or preferred outcomes of a variety of surgeries. The prior historical data, such as data that correlates prior bone product with a specific surgeon, is incorporated into the decision support platform in the functionality of the demonstrated system output. Where a surgeon is a user, for example, that surgeon may indicate (and the system store and record the respective data) the surgeon's inputs to better feed the decision tree outputs. More likely, however, is that a clinician supporting the surgeon, or a sales representative identifies and inputs the data corresponding to the product selected for the particular patient.

The recommendation of the optimal bone grafting material and/or procedure for the patient is output to the device of the provider (e.g., the surgeon) for display and/or stored. In some embodiments, the data output regarding the recommendation of the optimal bone grafting material and/or procedure for the patient can include data regarding the optimal bone grafting material (e.g., volume, texture, hydration, material, bone growth factor, such as BMP2, etc.), the optimal surgical method of insertion, the projected clinical outcomes, regulatory compliance data, pre-operation planning (e.g., pre-habilitation), post-operative care (e.g., rehabilitation, follow-up procedures, pain medication, steroids, medications, etc.), surgery details (e.g., procedure type, implant type, time, anesthesia, etc.), regulatory compliance data, and any other data regarding the materials or procedure.

Any patient wearable data may also feed directly into the inputs of the system, or indirectly through a patient user interface (UI). In some embodiments, data regarding the recommendation of the bone grafting material and/or procedure is output to an application on the device of the patient upon approval by the provider (e.g., the surgeon). Wearable data then can be incorporated as authorized by a patient. For example, the output data can include pre-operation planning, surgery details, post-surgical review, follow-up procedures, etc., specifically as to implants, materials used, pharmacologics or drugs administered, instruments utilized (e.g. what medical device and fixation devices can be recorded to facilitate removal of the device and/or components in a removal or redo procedure). Names of surgeons, nursing and medical staff within the operating room (OR) can be recorded, drug reactions, anesthesiologist and anesthesia protocol may also be useful upon patients seeking additional health care and surgical procedures.

In some embodiments, the provider can utilize the recommendation to manually prepare the bone grafting material in a device, such as a syringe, for injection of the bone graft into the patient during the procedure. In some embodiments, the provider can select the recommendation in the application in order to cause bone graft material preparation hardware in communication with the application to automatically prepare the bone grafting material. In some embodiments, the provider can select the recommendation in the application in order to cause bone graft material preparation hardware in communication with the application to automatically prepare the bone grafting material and automatically load the prepared bone grafting material in a robotic system, such as a robotic arm system, to assist the surgeon in performing the bone graft procedure. Imaging of the patient bones may identify the location and volume for bone autograft removal, region of bone degeneration, and facilitate analytical processing in determining the bone graft material and amount.

In the context of bone graft preparation hardware, and automating the same, the scrub tech or surgeon at the back-table would “formulate or make” the bone graft in a selected display with material implant selections. In such cases, the various off-the-shelf products would be selected, prepped, and/or hydrated, then combined as needed with patient-derived bone autograft, as additionally milled/refined, mixed and packaged into a canula or other syringe, mesh, capsule, or other insertion device. In some embodiments, (not illustrated) the autograft is cleaned and/or refined and milled into smaller particulates to best mix with the off-the-shelf bone grafts, including use of any bone mill or similar mobile milling device. Manual or automated process here may be incorporated as selected at the design user interface. Based upon the factors of patient health and product implant characteristics, the specific “recipe” for the bone graft mixture will be recommended and refined by surgeon or clinician selection (e.g. 2 parts autograft to 1 part INFUSE; or 3 parts DBM: 1 part autograft particulate, with refined milling to micrometer or millimeter dimensions, and hydrated with glycerol solution no more than 10%), or wash with saline and package). A variety of instructions can be indicated and refined appropriately.

In another embodiment, cannulas and meshes or any capsule or containment vessel can be manufactured by 3D printing or advanced manufacturing at the back-table in combination with the implant, bone graft, and or biologics selection.

In some embodiments, the neural network is trained to determine a recommendation of a bone graft material and/or procedure that optimizes the product selection with the particular patient characteristics, the bone graft characteristics (e.g., osteoconduction, osteoinduction, and osteogenesis), and related osteogenic capacity score of product and/or patient as based on the desired clinical outcome (i.e. U.S. procedures focus on fusion rates; European procedures also characterize pain and otherwise) and/or minimize cost via finance based records of prior surgeries regarding bone graft procedures. In some embodiments, the clinical outcome can be maximized based on a function of the bone graft (e.g., the osteoconduction, osteoinduction, and/or osteogenesis of materials of the bone graft), the osteogenic capacity of the patient (e.g., health data of the patient and/or location of the bone graft), and/or the bone graft procedure (e.g., the surgeon's experience and/or tools utilized by the surgeon for the bone graft procedure) via historical data regarding bone graft procedures. In some embodiments, the cost can be minimized as a function of the cost of the bone graft procedure, surgical time, rehabilitation time, probability of adverse events, probability of surgical revisions, patient satisfaction, quality of life, postoperative return to work, and/or any other factors via historical data regarding bone graft procedures.

In another aspect, artificial intelligence (AI) models, which can include machine learning models and generative AI models or train on data to create desired outcomes. An AI model program has been trained on a set of data to recognize certain patterns or make certain decisions without further human intervention; the initial training, however, is encoded by the human developer. Artificial intelligence models apply different algorithms to relevant data inputs to achieve the tasks or the output of which they have been programmed. Both machine learning (ML) and AI learn from data but the strategies differ. AI has now been interpreted as the broad, encompassing concept, while ML learns patterns from data, dep learning (DL) leverages deep neural networks for intricate pattern recognition, prediction, or classification; and generative AI creates new content. For clarification, AI has human coded trained algorithms to train the code to recognize certain patterns and make particular decisions; machine learning, as used here is a learning of the pattern and correlations without human interference, and objectively identified correlations, if any exist; deep learning is a subset of machine learning. For instance, machine learning is focused on analyzing data to find patterns and make accurate predictions, while generative AI, on the other hand, can be used to create new data that resembles training data. As such, for purposes here, ML or AI, which may include generative AI, are utilized, as appropriate. For example, particular models and implementations may be utilized as based on the desired objective or purpose, client, intended users, and/or risks identified. By way of example, technologies for a digital twin of a patient are provided herein, which, according to various embodiments, utilize AI models. The AI models can be trained to resemble data that is identified through the ML patterns.

In one aspect, a physiology-informed model is deterministic. Here, the clinical outcome of a bone graft procedure can be given by the following equation:

$$Outcome = [ f ( Bone Graft ) ] * [ f ( Patient ) ] * [ f ( Site ) ] * [ f ( Surgeon ) ] * [ f ( Hardware ) ] * [ f ( Procedure ) ] \\ where \\ [ f ( Bone Graft ) ] = [ {x}_{0} {OC}^{{y}_{0}} + {x}_{1} {OI}^{{y}_{1}} + {x}_{3} {OG}^{{y}_{2}} ]$$

Here, x₀OC^{y}^{₀} is generally based on the scaffold and refers to the osteoconduction (“OC”) of the bone graft material. The function x₀OC^{y}^{₀} can be based on a function of mineral, collagen/carrier, local bone, ICBG, porosity/space/architecture, resorption, etc. In some instances, if OC=0, no bone formation is contributed by the bone graft. Here, y₀=activity, which can be estimated using, for example, an athymic rat model.

Further, x₀OI^{y}^{₁} is generally based on the signal and refers to the osteoinduction (“OI”) of the bone graft material. The function x₀OI^{y}^{₁} can be based on a function of OI score, surface topography, ions, peptides, growth factors (BMP-2, etc.), etc. In some instances, if OI=0, no bone formation is contributed by the bone graft. Here, y₁=activity, which can be estimated using, for example, an athymic rat model.

Further, x₀OG^{y}^{₂} is generally based on the cells and refers to the osteogenesis (“OG”) of the bone graft material. The function x₀OI^{y}^{₁} can be based on a function of chemotaxis, BMA, autograft, stem cells, bone cells, other cells, etc. In some instances, if OG=0, no bone formation is contributed by the bone graft. Here, y₂=activity, which can be estimated using, for example, an athymic rat model. A ranking scale of 0-4 is utilized to characterize osteogenic capacity.

Further, [ƒ(Patient)]*[ƒ(Site)] refers to the osteogenic capacity of the location of the bone graft procedure for the specific patient. For example, [ƒ(Patient)] can be based on the pre-habilitation procedures, rehabilitation procedures, vitamin D, smoking, age, sex, non-steroidal anti-inflammatory drugs (NSAID) use, etc. of the specific patient and [ƒ(Site)] can be based on blood supply, surgical time, infection, bone quality, access, etc. of the specific location of the bone graft procedure.

Further, [ƒ(Surgeon)]*[ƒ(Hardware)] refers to the specific bone graft procedure. For example, [ƒ(Surgeon)] can be based on the surgeon's prior experience, pre-planning, back table, surgical approach, etc. of the specific surgeon and [ƒ(Hardware)] can be based on fixation quality, orientation, lordosis, anatomical fit, robotics, navigation, etc. utilized for the bone graft procedure.

In some embodiments, ƒ(bone graft) is an additive function. Not every bone graft is going to have all three areas (osteoconduction, osteoinductivity, and osteogenesis) but that is appropriate. For example, Infuse is highly osteoinductive, mildly osteoconductive, and technically not osteogenic (even though its mechanism influences chemotaxis of cells). In another example, synthetics would by highly osteoconductive, not osteoinductive, and osteogenic if combined with bone marrow aspirate. As such, the model is integrated and modified based on the factors selected and optimized based on clinically appropriate recommendations.

In some embodiments, the costs and/or insurance information of prior graft procedures are utilized to minimize the total cost of the surgery. In some embodiments, historical data regarding bone graft materials and procedures, historical EHRs for patients, and historical provider data are utilized to minimize the total cost of the surgery. In some embodiments, the application provides a selection for the user to select whether to only provide recommendations that optimize the clinical outcome of the surgery, only provide recommendations that minimize the cost of the surgery (e.g., and a selection of the type of cost, such as the total cost of the surgery based on insurance coverage), or any combination thereof.

In this regard, historical data regarding bone graft materials and procedures, historical EHRs for patients, and historical provider data can be utilized to train the neural network to recommend graft materials and/or procedures that optimize the clinical outcome and/or minimize the cost of the procedure.

Some embodiments are described with respect to an artificial neural network, a type of machine-learning model that learns to approximate unknown functions by analyzing example (e.g., training) data at different levels of abstraction. Generally, neural networks model complex non-linear relationships by generating hidden vector outputs along a sequence of inputs. In some cases, a neural network includes a model of interconnected digital neurons that communicate and learn to approximate complex functions and generate outputs based on a plurality of inputs provided to the model. In various implementations, a neural network includes any of a variety of deep learning models, including convolutional neural networks, recurrent neural networks, deep neural networks, and deep stacking networks, to name a few examples. In some embodiments, a neural network includes or otherwise makes use of one or more machine learning algorithms to learn from training data. In other words, a neural network can include an algorithm that implements deep learning techniques such as machine learning to attempt to model high-level abstractions in data. Although some implementations are described with respect to neural networks, some embodiments are implemented using other types of machine learning model(s), such as those using linear regression, logistic regression, decision trees, support vector machines (SVM), Naïve Bayes, k-nearest neighbor (KNN), K means clustering, random forest, dimensionality reduction algorithms, gradient boosting algorithms, neural networks (e.g., auto-encoders, convolutional, recurrent, perceptrons, Long/Short Term Memory (LSTM), Hopfield, Boltzmann, deep belief, deconvolutional, generative adversarial, liquid state machine, etc.), and/or other types of machine learning models.

### Overview of Technical Problems, Technical Solutions, and Technological Improvements

As described above, a spinal implant and a bone graft are typically selected by a spine surgeon in planning for a surgical procedure. The spinal implant is determined based on the anatomy of a patient and surgeon preference based on physical implant characteristics, design of the device, anatomical area of the spine for repair, and any surface topography that may assist in securing the implant at the implant location, or for use with biologics. A bone graft is commonly used in a variety of spinal surgeries, often used in about 50% or more of spinal implant surgeries; the procedure involves transplanting bone tissue, allograft, synthetic, autograft, or a mix to repair bones damaged from trauma (e.g., spinal fusion, bone fractures, etc.), assist bone healing or growth around surgically implanted devices (e.g., joint replacements, dental implants, etc.), or replace missing bone for any reason, such as injury, infection or disease. A significant number of materials within the biologics (e.g., natural or bioengineered substances), and combinations of materials, are available for bone graft procedures to promote bone healing. Unrealized compositions and combinations, however, remain open for configuration and design as based on personalized to a patient's needs and compatibility of the bone graft product. A surgeon will utilize their experience to choose the materials for the bone graft procedure based on factors, such as the size and location of the area to be treated, the patient's condition, and the surgeon's preferences. However, the surgeon may not be aware of the efficacy, or biocompatibility, of all bone graft materials and combinations of bone graft materials with respect to a specific procedure that involves a bone graft and specific health characteristics of the patient.

Currently, to select a bone graft product for a specific procedure, the surgeon must manually assess and analyze the patient's unique health history and the constantly increasing number of bone grafting materials. Further, if the surgeon would like to determine the efficacy of the bone grafting materials the surgeon must manually analyze similar surgical procedures for similar patients for each of the bone grafting materials to determine the efficacy of each of the bone grafting materials for the patient's unique health history. Time limitations, however, prevent this efficacy determination, and surgeons often rely on prior clinical/surgical procedures performed and their own patient outcomes. Accordingly, unnecessary personnel resources are utilized in conventional implementations that require manual analysis of the patient's unique health history, support by a sales representative and his/her knowledge of each bone grafting product, and/or each similar surgical procedure using each bone grafting product. For example, the staff operations related to the manual analysis of the patient's unique health history, each bone grafting product, and/or each similar surgical procedure using each bone grafting product unnecessarily consumes resources and expenses.

Advantageously, efficiencies of computing and network resource utilization can be enhanced using implementations described herein. In particular, the automated determination of the optimal bone grafting products and/or procedures provides for a more efficient use of computing and network resources than conventional methods of manually analyzing the patient's unique health history, each bone grafting product, and each similar surgical procedure using each bone grafting product. The technology described herein decreases the number of computer input/output operations related to manual analysis of the patient's unique health history, each bone grafting product, and each similar surgical procedure using each bone grafting product, thereby decreasing computation costs and decreasing network resource utilization (e.g., higher throughput, lower latency, and decreasing packet generation costs due to fewer packets being sent) when the information is located over a computer network.

### Additional Description of the Embodiments

Having briefly described an overview of embodiments of the technology described herein, an example operating environment in which embodiments of the technology described herein may be implemented is described below in order to provide a general context for various embodiments.

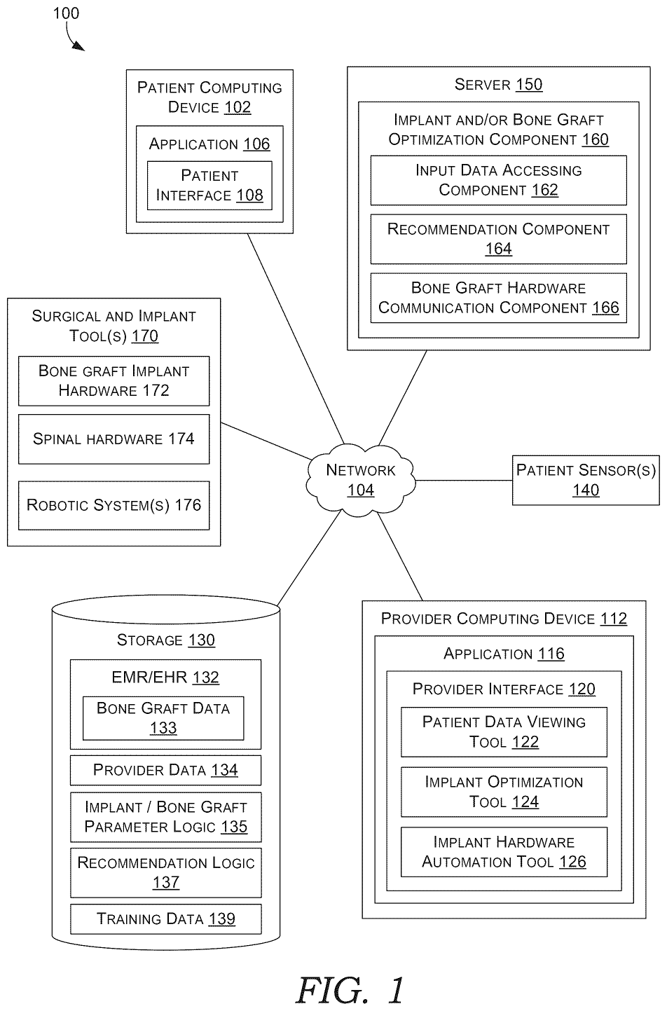

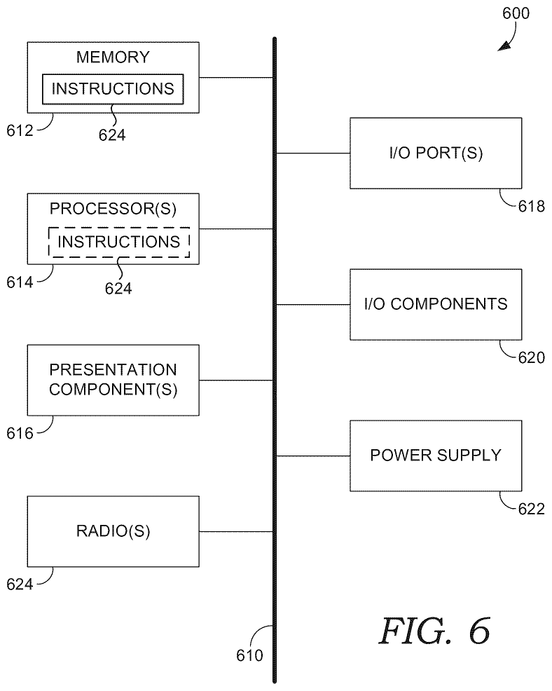

Turning to FIG. 1, a block diagram of example environment 100 suitable for use in implementing embodiments of the present disclosure is shown. Generally, environment 100 is suitable for using a machine learning computing system, such as an artificial neural network, trained on data regarding bone graft materials and procedures to determine the optimal implant, tools or instruments, bone grafting materials and/or procedures for the specific patient or context. Example environment 100 includes a patient computing device 102, a provider computing device 112, server 150, storage 130, one or more patient sensor(s) 140, and surgical and implant tool(s) 170. In various embodiments, patient computing device 102, provider computing device 112, and server 150 comprise a computing device, such as computing device 600 described below with reference to FIG. 6. In various embodiments, patient sensor(s) 140, storage 130, and robotic system(s) 176, storage 130, and surgical and implant tool(s) 170 comprise a computing device or operate in conjunction with a computing device, such as computing device 600, of FIG. 6.

Examples of such computing devices include, without limitation, a personal computer (PC); a laptop computer; a mobile computing device or a mobile device; a smartphone; a smart speaker; a tablet computer; a smart watch; a wearable computer; an implanted computing device; a personal digital assistant (PDA) device; a music player or an MP3 player, a global positioning system (GPS) or device a video player; a handheld communications device; a gaming device; an entertainment system; a fitness device; an electronic audio or listening device; an augmented reality or virtual reality vision device; a vehicle computer system; an embedded system controller; a camera; a remote control; an electronic appliance; a consumer electronic device; a workstation; a telemetry station; a command computing center that centralizes operations including computing, sensing and monitoring activities in and around a surgical suite and/or patient room, or throughout a medical facility; or any combination of these delineated devices, or any other suitable computer device. The data from these systems utilized selectively, or aggregated in selected configurations, can be used integrally, in some embodiments, with the predictive analytics engine, the data integrations included as desired in the analytics and learned pattern recognitions of ML/DL and/or AI tools.

As shown in example environment 100, patient computing device 102, provider computing device 112, and server 150 each include subcomponents that are each identified by functions performed by the subcomponent. In some embodiments, functions performed by these subcomponents (or functions performed by other components or subcomponents of FIG. 1) are associated with one or more computer applications, services, or routines, such as application 106 or application 116. These computer applications, services, or routines may operate on one or more computing devices (such as patient computing device 102, provider computing device 112, or server 150), or may be distributed across multiple computing devices or in the cloud. Moreover, these components, functions performed by these components, or services carried out by these components may be implemented at appropriate abstraction layer(s) such as the operating system layer, application layer, hardware layer, etc., of the computing system(s). Alternatively, or in addition, the functionality of these components and/or the embodiments described herein can be performed, at least in part, by one or more hardware logic components. For example, and without limitation, illustrative types of hardware logic components that can be used include Field-Programmable Gate Arrays (FPGAs), Application-Specific Integrated Circuits (ASICs), Application-Specific Standard Products (ASSPs), System-on-a-Chip systems (SOCs), Complex Programmable Logic Devices (CPLDs), etc. Additionally, although functionality is described herein with regard to specific components shown in example environment 100, it is contemplated that in some embodiments, functionality of these components can be shared or distributed across other components.

Many components of environment 100 are communicatively coupled and in some implementations communicate with each other via a network 104. In some embodiments, network 104 includes one or more local area networks (LANs), wide area networks (WANs), and/or other networks. Such networking environments are commonplace in offices, enterprise-wide computer networks, intranets, and the Internet.

Example environment 100 includes storage 130. Storage 130 generally stores information including data, records, computer instructions (e.g., software program instructions, routines, or services), logic, profiles, and/or models used in embodiments described herein. For example, as shown in example environment 100, storage 130 stores EMR/EHR 132, which may include bone graft data 133; provider data 134; implant/bone graft parameter logic 135; recommendation logic 137; and training data 139. In an embodiment, storage 130 comprises a data store (or computer data memory). Further, although depicted as a single data store component, storage 130 may be embodied as one or more data stores or distributed data storage, such as a cloud storage system. Furthermore it is contemplated that information depicted as stored on storage 130 in FIG. 1, may be stored on include computer storage media of other components of environment 100, such as computer memory of patient computing device 102, provider computing device 112, server 150, or patient sensor(s) 140. In some embodiments, the information stored via storage 130 is accessible (or otherwise available) to other components of example environment 100.

In various implementations, the components of environment 100 include computer storage media, such as represented by storage 130 in environment 100, that stores information including data, data structures, computer instructions (e.g., software program instructions, routines, or services), and/or models (e.g., neural networks, such as machine learning models) used in some embodiments of the technologies described herein. For example, in some implementations, patient computing device 102, provider computing device 112, storage 130, patient sensor(s) 140, surgical and implant tool(s) 170, and server 150 comprise one or more data stores (or computer data memory). Further, although patient computing device 102, provider computing device 112, storage 130, patient sensor(s) 140, surgical and implant tool(s) 170, and server 150 are each depicted as a single component in FIG. 1, in some embodiments, patient computing device 102, provider computing device 112, storage 130, patient sensor(s) 140, surgical and implant tool(s) 170, and/or server 150 are implemented using any number of data stores, and/or are implemented using cloud storage.

Example environment 100 includes a provider computing device 112, which is generally a user computing device for use by a healthcare provider, such as a surgeon performing a procedure on a patient. Provider computing device 112 includes a provider interface 120, which may be embodied as a GUI, voice/audio-UI, or other UI operated by application 116, which comprises a software application or a set of applications. As shown in example environment 100, application 116 operating provider interface 120 comprises a number of application tools, which are further described herein, including a patient data viewing tool 122, an implant optimization tool 124, and an implant hardware automation tool 126. In accordance with embodiments presented herein, application 116 and/or provider interface 120 or its subcomponents, facilitates accessing and receiving information from a healthcare provider about a specific patient or a set of patients for which a recommendation of optimal implant or bone graft materials and/or procedures are determined. Embodiments of provider computing device 112, or its subcomponents, also facilitate accessing and receiving information from a healthcare provider about a specific patient or population of patients including patient history; healthcare resource data; physiological variables (e.g., vital signs), measurements, time series, predictions (including plotting or displaying the determined outcome and/or issuing an alert) described herein; or other health-related information. The provider computing device 112, or its subcomponents, further facilitates display of results, recommendations, or orders, for example. In an embodiment, provider computing device 112, or its subcomponents, facilitates receiving orders for a patient based on the results of monitoring and predictions, such as predicted outcomes on a virtual “twin” patient similar to a particular patient. Provider computing device 112 may also be used for providing diagnostic services or evaluation of the performance of the technology described herein in conjunction with various embodiments.

Example environment 100 includes a patient computing device 102, which is generally a user computing device for use by a patent, such as described in connection with computing device 600 of FIG. 6. Patient computing device 102 includes a patient interface 108, which may be embodied as a GUI or other UI operated by application 106, which comprises a software application or a set of applications. In some embodiments, patient computing device 102 comprises a mobile device used by the patient, such as the patient's mobile phone, and application 106 comprises an app that is installed on the user's mobile device.

Embodiments of applications 106 and 116 generally comprise a software application or a set of applications (which may include programs, routines, functions, or computer-performed services) residing on a client computing device, one or more servers in the cloud, distributed in the cloud environment, or on a client computing device such as a personal computer, a laptop, a smartphone, a tablet, a mobile computing device, or front-end terminals in communication with back-end computing systems. In an embodiment, applications 106 and 116 comprise a Web-based application or applet, or a set of applications usable to manage user services provided by an embodiment of the present disclosure. For example, in an embodiment, each of the applications 106 and 116 facilitates processing, interpreting, accessing, storing, retrieving, and communicating information acquired from patient computing device 102, patient sensor(s) 140, EMR/EHR 132, provider data 134, implant and/or bone graft optimization component 160, or storage 130, including predictions and evaluations determined by embodiments of the present disclosure.

Accessing and/or utilizing information through applications 106 and 116 or utilizing associated functionality may require a user, such as a patient or a clinician, to login with credentials. In another aspect, passwordless authentication systems, i.e., biometrics, or OKTA, multi-factor authentication, trustless authentication and the like may be utilized. Further, applications 106 and 116 may store and transmit data in accordance with privacy settings defined by clinician, patient, an associated healthcare facility or system, and/or applicable local and federal rules and regulations regarding protecting health information, such as Health Insurance Portability and Accountability Act (HIPAA) rules and regulations.

In an embodiment, applications 106 and 116 can send a notification (such as an alarm or other indication) directly to provider computing device 112 or patient computing device 102 through network 104. Applications 106 and 116 may also send maintenance indications to provider computing device 112 or patient computing device 102. Further, an interface component may be used in applications 106 and 116 to facilitate access by a user (including a healthcare provider or patient) to functions or information on patient sensor(s) 140, such as operational settings or parameters, user identification, user data stored on patient sensor(s) 140, and diagnostic services or firmware updates for patient sensor(s) 140, for example.

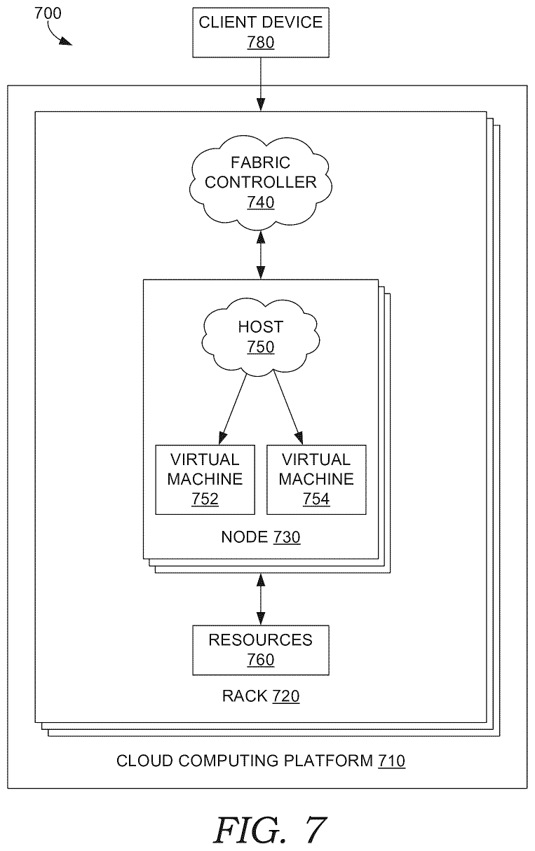

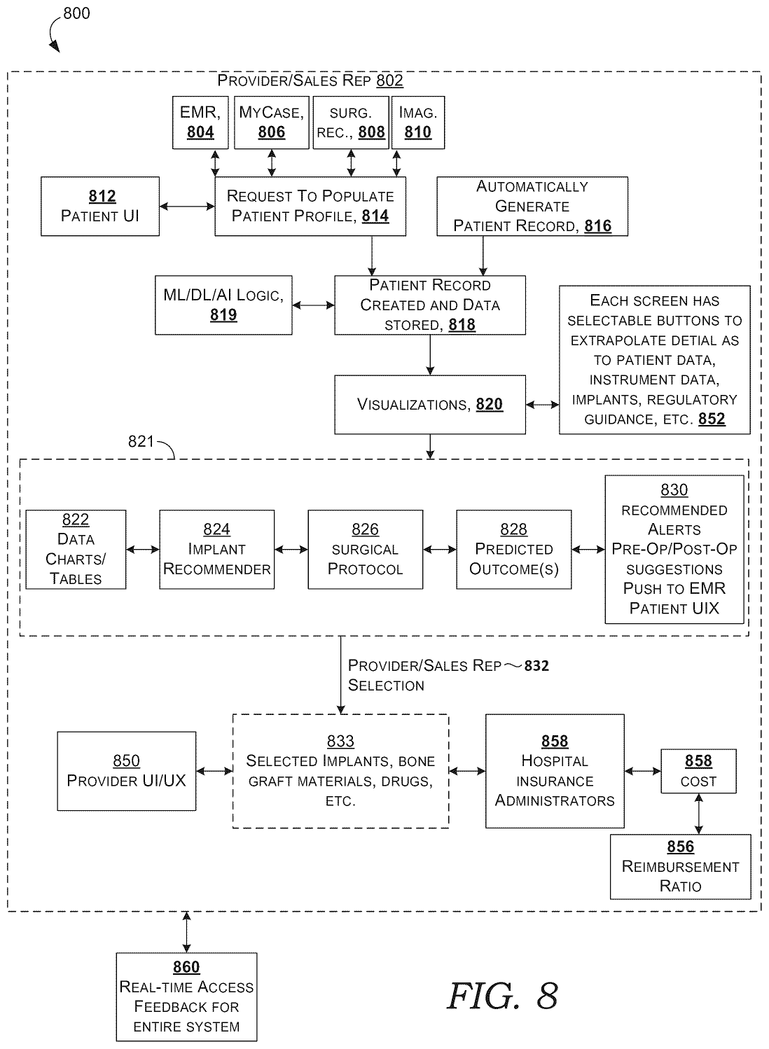

Example environment 100 also includes a server 150, which comprises a single computing device or multiple computing devices, such as described in connection with computing device 600 in FIG. 6, or may be embodied as a distributed computing environment such as cloud computing platform 710, described in FIG. 7. Server 150 includes implant and/or bone graft optimization component 160. In various embodiments, implant and/or bone graft optimization component 160 comprises one or more computing applications or computing services that facilitate determining a recommendation for a bone graft material and/or procedure, as further described herein. As shown in example environment 100, implant and/or bone graft optimization component 160 subcomponents, further described herein, including an input data accessing component 162, a recommendation component 164, and a bone graft hardware communication component 166.

In various embodiments, patient interface 108, provider interface 120, implant and/or bone graft optimization component 160, and/or any of the elements illustrated in FIG. 1 are incorporated, or integrated, into an application(s) (e.g., a corresponding application on patient computing device 102, provider computing device 112, and/or server 150, respectively), or an add-on(s) or plug-in(s) to an application(s). Moreover, it is contemplated that server 150 may, at times, operate as a client device, and other computing devices of environment 100, such as patient computing device 102, provider computing device 112, or storage 130 may, at times, operate as a server; for instance, to serve up information in response to a request from another device. In some embodiments, the application(s) 106 and/or 116 is any application capable of facilitating data regarding the patient, surgical procedures and/or materials, such as a stand-alone application, a mobile application, a web application, and/or the like. In some implementations, the application(s) 106 and/or 116 comprises a web application, for example, that may be accessible through a web browser, hosted at least partially server-side, and/or the like.

In various embodiments, the functionality described herein is allocated across any number of devices. For example, in some embodiments, application(s) 106 and/or 116 are hosted at least partially server-side, such that patient interface 108, provider interface 120, implant and/or bone graft optimization component 160, and/or any of the elements illustrated in FIG. 1 coordinate (e.g., via network 104) to perform the functionality described herein. In another example, provider interface 120, implant and/or bone graft optimization component 160, and/or any of the elements illustrated in FIG. 1 (or some portion thereof) are integrated into a common application executable on a single device. Although some embodiments are described with respect to an application(s), in some embodiments, any of the functionality described herein is additionally or alternatively integrated into an operating system (e.g., as a service), a server (e.g., a remote server), a distributed computing environment (e.g., as a cloud service), and/or otherwise. These are just examples, and any suitable allocation of functionality among these or other devices may be implemented within the scope of the present disclosure.

Example environment 100 also includes patient sensor(s) 140. As used herein, a sensor may include a function, routine, component, or combination thereof for sensing, detecting, or otherwise obtaining information such as data regarding a patient, and may be embodied as hardware, software, or combination of hardware and software. A0012183 Patient data that is acquired via a patient sensor 140 may be communicated to other specific components of environment 100 that operate on patient-related data, as described herein, or may be stored in a record associated with the patient, such as in EMR/EHR 132 of storage 130.

In some embodiments, a patient sensor 140 may comprise one or more sensors and/or computing components for storing sensed data and processing it to output interpreted data. For example a location sensor may comprise a sensor for detecting GPS data, a sensor for detecting cellular communication data, and a sensor for detecting Wi-Fi, as well as computing components for processing the sensed data determine location. In this example, the data outputted from the location sensor (i.e., location data) represents interpreted data based on the sensed and processed GPS or wireless communications data. In this regard, the data provided by a patient sensor 140 may comprises raw, unprocessed data, processed (or pre-processed data, e.g. normalized data) or interpreted data. For example a patient sensor 140 for providing temperature of a patient may output: a digital signal corresponding to a sampled temperature of the patient, a time series of multiple measurements obtained over time, of the patient's temperature, or an average (or mean) temperature for the patient over a window of time. Similarly another patient sensor 140 may output interpreted data that is location data derived from detecting other data such as GPS, Wi-Fi, or cellular network data.

In some implementations, a user computing device, such as patient computing device 102, provider computing device 112, or other computing device is configured to be used as a patient sensor 140 for acquiring information regarding a patient. These user devices may comprise one or more sensors, or may be used in conjunction with one or more sensors, such as patient sensor(s) 140. For example, the user device may comprise a wearable computing device or smart device (e.g. an Apple watch running an app for monitoring patient health data, or other wearable device, an earbud monitoring device that senses motion, balance, posture and walking gait, or the like). One embodiment of patient sensor(s) 140 comprises a user device that is a wearable wrist computing device with an integrated sensor, such as a smart watch or a tablet that is communicatively coupled to a source of sensor data. In some embodiments, the user device may include an integrated patient sensor 140) or operate in conjunction with external sensor.

In some aspects, patient sensor(s) 140 may be positioned on or near the monitored patient's wrist. It is contemplated that patient sensor(s) 140 may alternatively be positioned on or near an appendage (e.g., on or near the user's head, attached to the subject's clothing, worn around the subject's head, neck, leg, arm, ankle, finger, etc.). In other aspects, patient sensor(s) 140 may comprise a skin-patch sensor attached or adhered to the subject's skin or clothing; ingestible or sub-dermal sensor; sensor components integrated into the subject's living environment (including the bed, pillow, or bathroom); and sensors operable with or through a smartphone carried by the subject, for example. In some embodiments, patient sensor(s) 140 comprise wearable body sensors such as sensors operating in connection with a spinal wearable brace, band, or torso wrap configured to sense the flexion and movement of a patient; or an earbud device configured for sensing acoustic data or other data such as position or motion (e.g., posture and balance), for determining or inferring brain activity a patient.

In some embodiments, patient sensor(s) 140 comprise one or more gyroscopic sensors, accelerometer sensors, or similar sensors for detecting motion information. For example, patient sensor(s) 140 may comprise a wearable accelerometer sensor, which may be implemented on a fitness tracker wristband device, a smartwatch, and/or a smart mobile device such as a mobile phone running an app. Other types of sensors may also be integrated into or work in conjunction with user devices, such as sensors configured to detect ambient light (e.g., a photodetector); sensors configured to detect user location (e.g., an indoor positioning system (IPS) or a global positioning system (GPS)); sensors configured to detect atmospheric information (e.g., a thermometer, a hygrometer or a barometer); and physiological sensors (e.g., sensors detecting heart rate, blood pressure, core body temperature, near body temperature, galvanic skin response (GSR), or vitals). Some embodiments include multiple patient sensors 140, such as three sensors, to obtain accelerometer data, ambient light data, and temperature (e.g., near-body temperature) data. Some embodiments of patient sensor(s) 140 may include sensors measuring information to be used to monitor fine eye movement or fine finger movement, such as electromyography (EMG) for measuring activation of muscles, acoustic surveillance, and/or vibration transducers. It is contemplated, however, that physiological information about a patient or other patient-related data, which may include historic or contemporary data about the patient, according to embodiments of the disclosure, may also be received from an electronic medical record (EMR) or other electronic health record (EHR) corresponding to the patient, such as EMR/EHR 132, or from human measurements or human observations.

In some embodiments, patient sensor(s) 140 comprise a medical instrument or device for sensing or otherwise obtaining information about a patient, such as without limitation, a device used to produce ECG or EKG, MRI, CT scan, x-ray imaging, PET scan; an imaging device including thermal imaging, sonogram, still and motion video camera devices; a wearable device such as described previously; bedside, home, or vehicle sensors, or the like. In some embodiments, patient sensor(s) 140 comprise procedures for measuring, determining, or inferring information about a patient including procedures that are automated in part or in full by a computer, or are facilitated by human performance, such as laboratory testing (for example, bloodwork, biomarker testing, pathology, patient assessments, including those self-performed by a patient, as well as computer-assisted technology (for example, a smart sensor or -software based sensor) for determining patient information based on other information obtained about the patient.

Data may be acquired by patient sensor(s) 140 continuously, periodically, as needed, or as it becomes available. Further, data acquired by patient sensor(s) 140 may be associated with time and date information and may be represented as one or more time series of measured variables. In an embodiment, patient sensor(s) 140 collects raw sensor information and performs signal processing, forming variable decision statistics, cumulative summing, trending, wavelet processing, thresholding, computational processing of decision statistics, logical processing of decision statistics, pre-processing, and/or signal condition. Alternatively, one or more of these functions may be performed by a user device, such as patient computing device 102 or provider computing device 112, server 150, and/or applications 106 or 116.

As mentioned above, operating environment 100 includes EMR/EHR 132 which may be stored on storage 130 and may be associated with a monitored individual, such as a patient. Although FIG. 1 depicts storage 130 as stored on a single storage component, as described previously, some embodiments of storage 130 comprise a plurality of storage locations, such as distributed storage or cloud storage. Thus aspects of EMR/EHR 132 may be stored in different locations including in memory of other components of operating environment 100. Moreover, embodiments of storage 130 may be directly or indirectly communicatively coupled to other components of operating environment 100, via network 104. Accordingly, these components may access information stored in EMR/EHR 132 via communicating with storage 130.

Generally EMR/EHR 132 comprises information regarding electronic health records or electronic medical records for one or a plurality of patients. However, although referred to herein as a medical record or health record, some embodiments of EMR/EHR 132 include any information regarding a patient. For example, in some instances, EMR/EHR 132 includes patient demographic information, insurance information, data regarding the patient's lifestyle, habits, behaviors, occupation, hobbies or interests, diet and fitness related data, contextual information regarding the patient, or any other data regarding the patient. In some embodiments, EMR/EHR 132 includes information about other patients or other individuals, such as individuals who are similar in some regard to a particular patient. In some embodiments, the information about other patients includes outcomes of surgical procedures or healthcare treatments for those individuals who are similar in some regard to the particular patient. Further still, some embodiments of EMR/EHR 132 include data regarding virtual clinical analyses (e.g. simulated or predicted outcomes) related to the patient such as data regarding a virtual patient that is similar to the particular patient (e.g. a virtual twin patient). In some embodiments, data that is acquired or processed via patient sensor(s) 140 is stored in EMR/EHR 132.

In some embodiments, EMR/EHR include bone graft data 133. Bone graft data 133 generally comprises information regarding a bone graft associated with a patient. In some embodiments, bone graft data 133 is predetermined based on a particular healthcare provider for the patient. Some embodiments of bone graft data 133 include information regarding bone graft materials or procedures for a patient that are determined according to embodiments of this disclosure. For example, specific bone graft materials or procedures regarding a patient that are provided by recommendation component 164 may be stored in bone graft data 133 of an EMR/EHR 132 associated with the patient. Alternatively or in addition, bone graft data 133 comprises information regarding candidate bone graft materials or procedures regarding a particular patient or set of patients. For instance, these candidate materials or procedures may comprise those materials available to the surgeon or approved for use on the patient, or those procedures for which the provider-surgeon is trained to perform. Some embodiments of bone graft data 133 comprise data regarding other patients similar to a particular patient and who have had bone grafts, or data regarding virtual clinical analyses (e.g. simulated or predicted outcomes) related to the patient such as data regarding a virtual patient that is similar to the particular patient (e.g. a virtual twin patient).

In embodiments herein, bone graft data be a function of the bone graft, ƒ(bone graft) above, and established through a combination of osteoinduction, osteoconduction, osteogenesis supported by product composition, known mechanism of action, and literature (pre-clinical and clinical). Such literature learnings can be incorporated into the model. The information from the literature can seed the model with some basic information leading to a rudimentary functioning system. This system would then learn through the addition of incremental data that either reinforces or disproves the original data set.

In some embodiments, EMR/EHR 132 represents health information from different sources and may be embodied as distinct records systems, such as separate EHR systems for different healthcare provider computing devices (such as 112). As a result, the healthcare provider computing devices may be for healthcare providers of different provider networks or care facilities.

Embodiments of EMR/EHR 132 include one or more data stores of patient-related data, such as health records, which may be stored on storage 130, and may further include one or more computers or servers that facilitate storing and retrieving health records. In some embodiments, EMR/EHR 132 may be implemented as a cloud-based platform or may be distributed across multiple physical locations. EMR/EHR 132 may further include record systems that store real-time or near real-time patient (or user) information, such as wearable, bedside, or in-home patient monitors, for example.

Continuing with FIG. 1, storage 130 of example operating environment 100 also includes provider data 134. Provider data 134 generally comprises healthcare-provider-related data such as associated with a surgeon involved in a procedure for a patient. Provider data 134 also may include data regarding the health care organization or institution, such as a hospital or hospital system; insurance; sales representative associated with a surgical and implant tool(s) 170 or material used for implants or bone graft; or a surgeon involved in a procedure concerning a patient. For example, provider data 134 can include any data regarding a surgeon, such as any historical preferences of the surgeon; prior experience of the surgeon, which may include the outcomes of prior surgical procedures (e.g., procedures involving bone grafts, procedures similar to the procedure selected for the patient, and/or etc.); time to perform the prior procedures; data regarding learning experiences regarding surgical procedures; and/or the like. In some embodiments, provider data 134 includes data regarding a group of surgeons (e.g., a group at a particular hospital or in a particular healthcare network).

Storage 130 also includes implant/bone graft parameter logic 135 and recommendation logic 137. As further described herein, some embodiments of this disclosure generate optimal bone graft parameters that are used to determine a recommendation for a particular patient regarding aspects of an implant or bone graft, such as materials and procedures. For example, some embodiments of implant and/or bone graft optimization component 160 (or a subcomponent) determine optimal bone graft parameters or a recommendation that is determined based on one or more of the optimal bone graft parameters. In some embodiments, implant/bone graft parameter logic 135 and recommendation logic 137 is used to determine an optimal bone graft parameter and recommendation, respectively.

Accordingly, implant/bone graft parameter logic 135 facilitates determining optimal bone graft parameters in accordance with embodiments of this disclosure. Embodiments of implant/bone graft parameter logic 135 comprise computer instructions including one or more rules, conditions, associations, models, or other criteria for, among other operations, determining one or more optimal bone graft parameters. Implant/bone graft parameter logic 135 may take different forms, for example, depending on the particular parameters being determined or depending the configuration of recommendation logic 137 that utilizes one or more bone graft parameters to determine a recommendation. For example, the form of implant/bone graft parameter logic 135 may configured to operate with a particular recommendation logic 137 that includes a particular trained neural network designed to operate according to certain bone graft parameters. Accordingly, implant/bone graft parameter logic 135 may comprise a set of rules, such as Boolean logic, various decision trees (e.g., random forest, gradient boosted trees, or similar decision algorithms), conditions or other logic, fuzzy logic, neural network, finite state machine, support vector machine, machine-learning techniques, or combinations of these to determine (or facilitate determining) one or more optimal bone graft parameters according to embodiments described herein. Some embodiments of implant/bone graft parameter logic 135 determine one or more bone graft parameters that correlate with parameters of a virtual patient in order to maximize the clinical outcome for a real patient.

Recommendation logic 137 facilitates determining a recommendation in accordance with embodiments of this disclosure. For example, as described herein, a recommendation may be determined for a patient regarding particular implants, bone graft materials, biologics, tools, and/or procedures regarding the patient. Various embodiments of recommendation logic 137 comprise computer instructions including one or more rules, conditions, associations, models, or other criteria for determining the recommendation. In some embodiments, recommendation logic 137 includes logic (such as computer-executable instructions) for programmatically determining a recommendation to be provided to a healthcare provider, such as a surgeon, regarding an aspect of surgery or treatment for a particular patient, such as a particular implant, bone graft material, biologic, tool, and/or procedure. Accordingly, embodiments of recommendation logic 137 may take different forms. For example, recommendation logic 137 may comprise a set of rules (which may include static or predefined rules or may be set based on settings or preferences (e.g., which may be stored in provider data 134) in a profile associated with the provider or a group profile associated with a group or providers or healthcare organization such as a hospital), Boolean logic, decision trees (e.g., random forest, gradient boosted trees, or similar decision algorithms), conditions or other logic, a deterministic or probabilistic classifier, fuzzy logic, neural network, finite state machine, support vector machine, logistic regression, clustering, machine learning techniques, prediction models, similar statistical processes, or combinations of these.

For example, some embodiments of recommendation logic 137 comprise a neural network that is trained to facilitate determining a recommendation of a bone graft material and/or procedure. In some instances, the neural network of recommendation logic 137 is trained or configured to determine a recommendation that optimizes the bone graft characteristics (e.g., osteoconduction, osteoinduction, and osteogenesis) of the implanted construct to maximize clinical outcome and/or minimize cost via historical data regarding bone graft procedures.

In particular, some embodiments of the trained neural network of recommendation logic 137 are configured to utilize patient-related data (e.g. information from EMR/EHR 132 regarding a particular patient); data regarding a surgeon designated to perform the procedure (e.g., provider data 134), which may comprise historical surgical data regarding the surgeon, and/or other data regarding optimal bone grafting parameters, which may include aspects of data regarding materials and/or procedures. In some embodiments, the trained neural network of recommendation logic 137 is utilized to determine a recommendation of an optimal bone grafting material, or aspects thereof, along with recommendations regarding an optimal procedure or aspects thereof. For example, the trained neural network of recommendation logic 137 may be used to generate a recommendation for an optimal implant, such as a titanium, carbon fiber, or a bio-compatible polymer cage for an interbody fusion or an extradiscal fixation device, for the procedure and/or bone grafting materials.

Storage 130 also includes training data 139. In various embodiments, training data 139 comprises data used for training, validating, generating, or configuring, a model that is included in some embodiments of implant/bone graft parameter logic 135 and recommendation logic 137. For instance, in the above example of recommendation logic 137 that comprises the trained neural network, training data 139 includes data used to train the model. In some embodiments, training data 139 includes any health data for the patient, such a but not limited to ECG or EKG, labs/pathology, wearable technology, sensors biometrics, bone health, calcium compositions, images/video/audio of the patient, data from genomics tools, such as a third party database, diagnostic records, blood testing, histology, blood pressure, heart rate, recurring status, compliance with medicines and prescribed treatment, activities, fusion rates, pain thresholds and assessments, age, weight, height, other biometrics, bone health, calcium compositions, personal demographic data, such as age, sex, and/or economic status, insurance coverage and/or payouts, diagnostics, social conditions, psychological conditions, physical conditions or stress conditions, geographical location blood work, blood pressure, pain, and health monitoring information, and any other information regarding the patient historical data, historical use or implant, bone graft and biologic, including prior bone graft procedures and outcomes data. Published clinical study data sets may also be used to establish initial algorithms or relationships.

Example operating environment 100 also includes a number of surgical and implant tool(s) 170, which in various embodiments, may comprise a computing device or operate with a computing device, as described previously. Examples of surgical and implant tools 170 include bone graft implant hardware 172; spinal hardware 174, such as surgical instruments, intradiscal and/or extradiscal implants and/or other instrumentation for implant procedures; and robotic system(s) 176, such as robotic guidance systems for performing spinal surgery.

With continuing reference to FIG. 1, several example workflows are provided to show by example the various operations carried out by the components of environment 100 in accordance with embodiments of this disclosure. A first example workflow of the configuration illustrated in FIG. 1 includes provider computing device 112, such as a desktop, laptop, or mobile device such as a tablet or smart phone, and application 116 provides one or more user interfaces. In some embodiments, a user-provider (e.g., a surgeon, doctor, nurse, counselor, health insurance administrator, medical specialist, medical materials or device manufacturer, supplier, sales representative, or other medical consultant for bone graft materials, tools, or aspects thereof) accesses aspects of patient data for a particular patient in provider interface 120 of application 116 via the patient data viewing tool 122. In some embodiments, the user selects a representation of a procedure involving a bone graft for a patient in provider interface 120 of application 116 via the implant optimization tool 124. In some embodiments, a recommendation of bone graft materials and/or procedure (e.g., as determined by implant and/or bone graft optimization component 160) is provided for a patient to a provider in provider interface 120 of application 116 via the implant optimization tool 124. In some embodiments, the user implements preparation of a bone graft material through surgical and implant tool(s) 170 for a patient in provider interface 120 of application 116 via an implant hardware automation tool 126.

Another example workflow of the configuration illustrated in FIG. 1 includes patient computing device 102, such as a desktop, laptop, or mobile device such as a tablet or smart phone, and application 106 provides one or more user interfaces. In some embodiments, a patient can view data regarding the surgical procedure approved by the provider (e.g., via application 116 on provider computing device 112) in patient interface 108 of application 106 operating on patient computing device 102.

In some embodiments and as further described herein, implant and/or bone graft optimization component 160 facilitates application of a trained artificial neural network to determine a recommendation of the optimal implant or bone grafting materials and/or procedures for a patient. Aspects of data for application to the neural network can be accessed via input data accessing component 162. In some embodiments, this data is utilized by recommendation component 164 to determine a recommendation of an implant or bone graft material and/or an aspect of the procedure. The recommendation can be displayed via implant optimization tool 124 of the provider computing device 112. The provider can utilize the recommendation displayed via implant optimization tool 124 to facilitate preparation of the bone grafting material into a surgical and implant tool, 170, such as a syringe, for injection of the bone graft into the patient during the procedure. In some embodiments, the provider can select the recommendation via the implant hardware automation tool 126 in order to cause surgical and implant tool(s) 170 in communication with bone graft hardware communication component 166 to automatically prepare the bone grafting material, for example, via bone graft implant hardware 172. Information and computer instructions regarding accessing data, generating or providing recommendations, communicating with bone graft hardware, and/or training a neural network can be stored in any suitable storage location, such as storage 130, provider computing device 112, server 150, some combination thereof, and/or other suitable locations.

In some embodiments, input data accessing component 162 facilitates accessing input data (e.g., EHRs 132 of the patient, provider data 134 of a surgeon performing or involved in the procedure, selections by the provider via implant optimization tool 124, or other related data) to determine a recommendation of the optimal bone grafting materials and/or procedures for a patient. For example, the provider (e.g., a surgeon, doctor, nurse, counselor, health insurance administrator, and/or medical specialist, such as a medical materials or device manufacturer, supplier, sales representative, or medical consultant for bone graft materials, tools, or aspects thereof) selects or otherwise determines a representation of a procedure involving a bone graft for a patient via implant optimization tool 124. In some embodiments, the surgeon selects the various characteristics of the procedure via implant optimization tool 124, such as the type of implants (e.g., a titanium, carbon fiber, or a bio-compatible polymer cage, an allograft interbody spacer, fixation devices, and any other device implanted during a surgical surgery that utilizes a bone graft), they type of bone graft materials available for the procedure (e.g., whether ICBG is available for the patient, access to specific autografts, allografts, or synthetic bone grafts), instruments available for the procedure (e.g., such as whether the surgeon has access to a robotic system for assisting with the bone graft procedure) and/or any preferences of the surgeon (e.g., such as particular grafting materials that the surgeon prefers).

In some embodiments, input data accessing component 162 facilitates accessing input data regarding the patient, such as the patient's EHRs 132. For example, the patient's EHRs 132 can include any data regarding the patient, such as imaging for the patient, such as thermal imaging, CT scans, MRI scans, X-RAYs, PET scan, ultrasound, and/or etc. Generally, the patient's EHRs can include any health data for the patient, such as an ECG or EKG, labs/pathology, wearable technology, such as sensors, biometrics, OI, bone health, calcium compositions, images/video/audio of the patient, data from genomics tools, such as a third party database, diagnostic records, blood testing, histology, blood pressure, heart rate, recurring status, compliance with medicines and prescribed treatment, activities, fusion rates, pain thresholds and assessments, age, weight, height, other biometrics, bone health, calcium compositions, personal demographic data, such as age, sex, and/or economic status, insurance coverage and/or payouts, diagnostics, social conditions, psychological conditions, physical conditions or stress conditions, geographical location, bone health, blood work, blood pressure, pain, and health monitoring information, and any other information regarding the patient.

In some embodiments, input data accessing component 162 facilitates accessing input data regarding the surgeon designated to perform the procedure from provider data 134, such as historical surgical data regarding the surgeon.

In some embodiments, input data accessing component 162 facilitates accessing input data regarding data regarding the user's selections. For example, a surgeon may select specific preferences for specific materials and/or procedures via implant optimization tool 124. As another example, a medical specialist, such as a medical materials or device manufacturer, supplier, sales representative, or medical consultant selects or otherwise provides a specific preference for particular product or group of products, such as a particular allograft or DBM product, CBM or VBM product, or synthetic graft, via implant optimization tool 124. In some embodiments, the preferences can be selected or otherwise provided for a single user, such as a single surgeon, or a group of users, such as a group of surgeons at a particular hospital or in a particular healthcare network, via implant optimization tool 124. In some embodiments, the preferences can be crowdsourced from many users, such as by presenting recommended preferences selected by most surgeons, via implant optimization tool 124. In some embodiments, implant optimization tool 124 may provide prior selections, such as previous settings/configurations, and can include with links to information about the previous cases so that the user can access the information regarding the previous cases.

Data regarding procedures, materials, patient data, provider data, and/or provider selections can be stored in any suitable storage location, such as storage 130 or other components of example environment 100.

In some embodiments, recommendation component 164 facilitates determining a recommendation of a bone graft material and/or procedure using recommendation logic 137 (which, as further described herein may involve using a machine learning system such as a trained neural network). In particular, the recommendation comprises optimal aspects or the materials and/or procedure, such as particular bone graft characteristics: osteoconduction, osteoinduction, and osteogenesis, physical and biochemical attributes, surface chemistries, nano-structural components, handling/malleability, healing attributes, etc. of the implanted construct in order to maximize clinical outcome for a particular patient based on prior procedural histories and such related data from prior cases, and/or minimize cost based on the corresponding case data regarding bone graft procedures. For example, the trained neural network of recommendation component 164 utilizes the patient's EHRs 132, historical surgical data regarding the surgeon designated to perform the bone graft procedure from provider data 134, and/or data regarding the user's selections (e.g., as selected video implant optimization tool 124) to determine the recommendation of the optimal bone grafting material and/or procedure for the patient. In some embodiments, the trained neural network of recommendation component 164 determines a recommendation of the optimal bone grafting material, along with recommendations regarding the optimal procedure personalized with a digital patient portfolio. For example, the trained neural network of recommendation component 164 can recommend an optimal implant, such as a titanium, carbon fiber, or a bio-compatible polymer cage for an interbody fusion or an extradiscal fixation device, for the procedure and/or bone grafting materials.

The recommendation of the optimal bone grafting material and/or procedure for the patient is output via recommendation component 164 to implant optimization tool 124 for display via the provider computing device 112. In some embodiments, the data output regarding the recommendation of the optimal bone grafting material and/or procedure for the patient via recommendation component 164 can include data regarding the optimal bone grafting material (e.g., volume, texture, hydration, material, bone growth factor, such as BMP2, etc.), the optimal surgical method of insertion (i.e. manual manipulation, via syringe, injection device/gun, or automated robotic insertion), the projected clinical outcomes, regulatory compliance data, pre-operation planning (e.g., pre-habilitation), post-operative care (e.g., rehabilitation, follow-up procedures, pain medication, steroids, medications, etc.), surgery details (e.g., procedure type, time, anesthesia, etc.), regulatory compliance data, and any other data regarding the materials or procedure. In some embodiments, data regarding the recommendation of the bone grafting material and/or procedure is output via recommendation component 164 to patient interface 108 of application 106 for display on patient computing device 102 upon approval by the provider (e.g., the surgeon) via application 116. For example, the data output via recommendation component 164 to patient interface 108 can include pre-operation planning, surgery details, post-surgical review, follow-up procedures, etc., including alerts and suggested activities as directed by a physician.

In some embodiments, the neural network (e.g., recommendation logic 137) of recommendation component 164 can be trained to determine a recommendation of a bone graft material and/or procedure that optimizes the implant components, perhaps an interbody with a particular bone graft, the bone graft characteristics of the implanted construct to maximize clinical outcome as driven by machine-learned aggregations of data correlated to a digital configuration of a patient (i.e., digital twin of a patient=aka “patient avatar”); cost can also be driven down in the use of historical data regarding bone graft procedures. In some embodiments, the clinical outcome can be maximized based on a function of the bone graft (e.g., the osteoconduction, osteoinduction, and/or osteogenesis of materials of the bone graft), the osteogenic capacity of the patient (e.g., health data of the patient and/or location of the bone graft), and/or the bone graft procedure (e.g., the surgeon's experience and/or tools utilized by the surgeon for the bone graft procedure) via historical data regarding bone graft procedures as stored in training data 139. In some embodiments, the cost can be minimized as a function of the cost of the bone graft procedure, surgical time, rehabilitation time, probability of adverse events, probability of surgical revisions, patient satisfaction, quality of life, postoperative return to work, and/or any other factors via historical data regarding bone graft procedures as stored in training data 139.

In one aspect, the clinical outcome of a bone graft procedure can be given by the following equation to augment the results of the trained neural network (e.g., recommendation logic 137) of recommendation component 164:

$$Outcome = [ f ( Bone Graft ) ] * [ f ( Patient ) ] * [ f ( Site ) ] * [ f ( Surgeon ) ] * [ f ( Hardware ) ] \\ where \\ [ f ( Bone Graft ) ] = [ {x}_{0} {OC}^{{y}_{0}} + {x}_{1} {OI}^{{y}_{1}} + {x}_{3} {OG}^{{y}_{2}} ]$$

Here, x₀OC^{y}^{₀} is generally based on the scaffold and refers to the osteoconduction (“OC”) of the bone graft material. The function x₀OC^{y}^{₀} can be based on a function of mineral, collagen/carrier, local bone, ICBG, porosity/space/architecture, resorption, etc. In some instances, if OC=0, no bone formation is contributed by the bone graft. Here, y₀=activity, which can be estimated using, for example, an athymic rat model. Known properties of the bone graft, whether supported from non-clinical or clinical data may be utilized. For example, the hardware component may be an interbody or extradiscal component or fixation device taking the form of a metal or plastic or combination thereof, including surface chemistries and topographies to facilitate biocompatibility and bone adherence/growth, respectively.

Further, x₀OI^{y}^{₁} is generally based on the signal and refers to the osteoinduction (“OI”) of the bone graft material. The function x₀OI^{y}^{₁} can be based on a function of OI score, surface topography, ions, peptides, growth factors (BMP-2, etc.), etc. In some instances, if OI=0, no bone formation is contributed by the bone graft. Here, y₁=activity, which can be estimated using, for example, an athymic rat model, and supporting non-clinical or clinical data.

Further, x₀OG^{y}^{₂} is generally based on the cells and refers to the osteogenesis (“OG”) of the bone graft material. The function x₀OI^{y}^{₁} can be based on a function of chemotaxis, BMA, autograft, stem cells, bone cells, other cells, etc. In some instances, if OG=0, no bone formation is contributed by the bone graft. Here, y₂=activity, which can be estimated using, for example, an athymic rat model, inclusion of BMA, known cellular component of a CBM, supporting non-clinical or clinical data.

Further, [ƒ(Patient)]*[ƒ(Site)] refers to the osteogenic capacity of the location of the bone graft procedure for the specific patient. For example, [ƒ(Patient)] can be based on the pre-habilitation procedures, rehabilitation procedures, vitamin D, smoking, age, sex, non-steroidal anti-inflammatory drugs (NSAID) use, etc. of the specific patient and [ƒ(Site)] can be based on blood supply, surgical time, infection, bone quality, access, etc. of the specific location of the bone graft procedure.

Further, [ƒ(Surgeon)]*[ƒ(Hardware)] refers to the specific bone graft procedure. For example, [ƒ(Surgeon)] can be based on the surgeon's prior experience, pre-planning, back table, surgical approach, etc. of the specific surgeon and [ƒ(Hardware)] can be based on fixation quality, orientation, lordosis, anatomical fit, robotics, navigation, etc. utilized for the bone graft procedure.

In some embodiments, the costs and/or insurance information of prior graft procedures are utilized to minimize the total cost of the surgery. In some embodiments, historical data regarding bone graft materials and procedures, historical EHRs for patients, and historical provider data are utilized to minimize the total cost of the surgery. In some embodiments, implant optimization tool 124 provides a selection for the user to select whether to only provide recommendations that optimize the clinical outcome of the surgery, only provide recommendations that minimize the cost of the surgery (e.g., and a selection of the type of cost, such as the total cost of the surgery based on insurance coverage), or any combination thereof.

In this regard, historical data regarding bone graft materials and procedures, historical EHRs for patients, and historical provider data can be utilized to train the neural network (e.g., recommendation logic 137) of recommendation component 164 to recommend graft materials and/or procedures that optimize the clinical outcome and/or minimize the cost of the procedure. Data regarding the recommendations, neural network of recommendation component 164 and/or the training of neural network of recommendation component 164 can be stored in any suitable storage location, such as storage 130, provider computing device 112, server 150, some combination thereof, and/or other locations, such as EMR/EHR 132, provider data 134, recommendation logic 137, implant/bone graft parameter logic 135, and training data 139.

In some embodiments, bone graft hardware communication component 166 communicates with surgical and implant tool(s) 170 to cause surgical and implant tool(s) 170 to automatically prepare the bone grafting material via bone graft implant hardware 172. For example, the provider can select the recommendation of the bone graft material via implant hardware automation tool 126 in order to cause surgical and implant tool(s) 170 in communication with bone graft hardware communication component 166 to automatically prepare the bone grafting material via bone graft implant hardware 172 of FIG. 1 and/or spinal hardware 174. In some embodiments, the provider can select the recommendation of the bone graft material via bone graft hardware automation tool 126 in order to cause surgical and implant tool(s) 170 in communication with bone graft hardware communication component 166 to automatically prepare the bone grafting material and automatically load the prepared bone grafting material in a robotic system 176 of surgical and implant tool(s) 170, such as a robotic arm system, to assist the surgeon in performing the bone graft procedure. Data regarding instructions for bone graft material preparation via surgical and implant tool(s) 170 can be stored in any suitable storage location, such as storage 130, provider computing device 112, server 150, some combination thereof, and/or other locations, such as implant/bone graft parameter logic 135.

With reference to FIG. 2, an example diagram is provided depicting aspects of a programmatic decision-making process 200 to determine a recommendation of the optimal bone grafting materials and/or procedures for a patient. As shown in FIG. 2, data is received by logic 206, which comprises a trained neural network of optimal bone graft materials and/or procedure recommendation. Logic 206 is embodied as recommendation logic 137, and in some further instances also embodied as implant/bone graft parameter logic 135, of FIG. 1, in some implementations, which is used by a computer program or function, such as recommendation component 164 of FIG. 1. In the example of FIG. 2, the input data received by logic 206 includes patient data 202 (e.g., EHRs 132 of FIG. 1) for a patient and provider data 204 (e.g., provider data 134 of FIG. 1). Patient data 202 includes, for example, imaging, ECG, labs/pathology, other EHR, data from wearable technology (e.g., patient sensor(s) 140 of FIG. 1), biometrics, OI factors, bone health, calcium compositions, images, video and/or audio of the patient, insurance and/or economic factors of the patient. Provider data 204 includes, for example, selections or preferences of the provider, based on the historical data and as input via implant optimization tool 124 of FIG. 1, prior experience of the surgeon designated to perform the procedure, and the available surgical instruments and devices of the surgeon. The trained neural network of optimal bone graft materials and/or procedure recommendation logic 206 utilizes the input data to determine the optimal bone graft materials and/or procedure recommendation 208. The optimal bone graft materials and/or procedure recommendation 208 is then provided to the surgeon 210 for display at a provider UI via an application (e.g., application 116 of provider computing device 112 of FIG. 1) and/or stored. The provider UI is configured to provide a quick summation of patient and selected products and implants in pre-surgical planning. In some embodiments, the surgeon 210 can utilize the recommendation to manually prepare the bone grafting material, or insert into a device, such as a syringe, or for injection gun of the bone graft into the patient during the procedure. In some embodiments, the provider can select an action element 212 in the application for the automated preparation of the recommendation in order to cause bone graft material preparation hardware in communication with the application to automatically prepare the bone grafting material. In some embodiments, the provider can select an action element 214 in the application for the automated preparation of the recommendation and loading on robotic system in order to cause bone graft material preparation hardware in communication with the application to automatically prepare the bone grafting material and automatically load the prepared bone grafting material in a robotic system, such as a robotic arm system, to assist the surgeon in performing the bone graft procedure. Additional mechanisms, securing of bone graft into cannulas and/or measuring volumes of bone graft and enabling a pressure relief valve in the robotic injection system will facilitate operational design.

In an aspect of one embodiment, functionality is provided for the surgeon to view, select, and/or change the plan displayed in 210. That change, as it pertains to the patient, information related to the patient, product, or otherwise, may then push back through the model an updated recommendation creating a feedback loop in the decision making model. For example, if the surgeon changes the bone graft from DBM product of FIG. 2B to a cellular bone matrix (CBM), or perhaps includes a combination with rhBMP-2, the model reassesses this new parameter and recommends a new plan based on the directed selection and choice made by the doctor/surgeon. Such recommendation may include parameters of re-mixing the product selection with autograft, or selecting hydration component and the like. In another aspect, the system presents a version where they treat with DBM vs where they treat with an Infuse™ product line including a collagen sponge plus rhBMP-2. Each model predicts the patient outcome(s) based on the particular personal health information of an identified patient and personalizes the use of a recommended implant and/or biologic. The surgeon has the opportunity to decide between the options, and select the implant based on his/her expertise, but also while translating an expeditious report-out or visualization of machine learned correlations of objective clinical data characterizing the use of a particular product with patients of similar characteristics, including such symptom presentation and/or treatments.

Returning to FIG. 1, in an example implementation, provider interface 120 of application 116 provides interface functionality that allows a user (e.g., a provider, such as a surgeon) to view patient data, select inputs and/or trigger optimal bone graft recommendations via implant and/or bone graft optimization component 160, and/or trigger bone graft hardware to prepare bone graft material through interactions via bone graft hardware communication component 166 with an interface controlled by application 116. Generally, provider interface 120 presents one or more interaction elements that provide various interaction modalities for selecting, navigating, chatting with patients, viewing patient data, selecting data for bone graft optimization, and/or triggering bone graft hardware to prepare bone graft material. In various embodiments, these tools are implemented using code that causes a presentation of a corresponding interaction element(s), and detects and interprets inputs interacting with the interaction element(s).

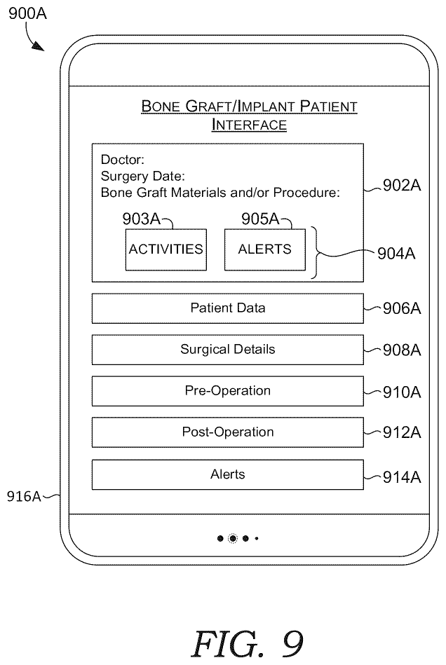

In the example implementation in FIG. 1, provider interface 120, comprises a computer user interface (UI), which in an embodiment comprises a GUI and further includes one or more tools for facilitating patient decision support. In particular, in the example implementation shown in FIG. 1, provider interface 120 includes a patient data viewing tool 122 that provides an interface to allow the provider to view patient data. Examples of an interface displayed to a provider (e.g., or a patient via patient interface 108 of application 106 of patient computing device 102) to view patient data are shown in FIGS. 3A and 3B. Further, in the example implementation in FIG. 1, provider interface 120 includes an implant optimization tool 124 that provides a user interface to allow the user-provider to select inputs and/or trigger optimal bone graft recommendations via implant and/or bone graft optimization component 160. An example of an interface displayed to a provider to trigger optimal bone graft recommendations are shown in FIG. 3C. An example of an interface displayed to a provider with optimal bone graft recommendations determined via implant and/or bone graft optimization component 160 are shown in FIG. 3D. Examples of an interface displayed to a provider with optimal bone graft recommendations determined via implant and/or bone graft optimization component 160, as well as information about the patient supporting the recommendation, surgical details, and other related information are shown in FIGS. 3D and 3E. In some implementations a patient interface 108 of application 106 provides interface functionality that allows a user (e.g., a patient) to view patient data and/or view data regarding a procedure involving a bone graft approved by the provider (e.g., surgeon) with an interface controlled by application 116. One example of a patient interface 108 is shown in FIG. 9 and further described in connection to FIG. 9. Such data may also include information about post-surgical guidance for the patient's recovery or scheduling of subsequent appointments (such as in-person or remotely performed telemedicine via the patient interface 108) for a care plan associated with the procedure. Generally, patient interface 108 presents one or more user-interface elements that provide various interaction modalities for selecting, navigating, chatting with providers, viewing patient data, and/or viewing data regarding the bone graft surgery.

In various embodiments, patient data viewing tool 122, implant optimization tool 124, implant hardware automation tool 126, and other aspects or provider interface 120, as well as patient interface 108 are implemented using computer instruction code that causes a presentation of a corresponding interaction element(s), and detects and interprets inputs interacting with the interaction element(s). For instance, in an embodiment of the example implementation in FIG. 1, patient interface 108 provides a GUI to allow a patient to view data about the patient and/or view data regarding the bone graft surgery. FIGS. 3A through 3E depict various screenshot illustrations of a provider UI, such as provider interface 120 of FIG. 1, whereby the buttons designed at the interface can be clicked and expanded to another window to detail the information requested by the user. Similarly, an example of an interface configured for use by a patient (such as patient interface 108 of application 106 of patient computing device 102) to view patient data and/or view data regarding the bone graft surgery is shown in FIG. 9.

### Example Bone Graft Recommendation Application Interface

FIGS. 3A through 3E and FIG. 9 depict various examples of GUIs 300A-300E and 900A, respectively, that are presented via a computing device. In particular, the user interfaces 300A-300E depicted in FIGS. 3A-3E are shown as being presented on a provider computing device 312 such as a tablet computer, and which may be implemented as described in connection with provider computing device 112 of FIG. 1. Interface 900A, depicted in FIG. 9, is shown as being presented on a patient computing device 916A, such as a tablet computer, and which may be implemented as described in connection with patient computing device 102 of FIG. 1.

However, it is also contemplated that these and other user interfaces described herein may be presented via a computing device used by the patient (e.g., patient computing device 102), an administrator, a sales person, another caregiver, or another user. Additionally, the same device, such as provider computing device 112, may present any or all of the user interfaces 300A-300E described in connection to FIGS. 3A through 3E. In an embodiment, each or some of user interfaces 300A-300E include a navigation tool 301 that enables the user of provider computing device 112 to navigate to the various user interfaces 300A-300E by swiping left or right on a touch screen of provider computing device 112, or my indicating to the user that other interfaces are available for presentation.

Turning first to FIG. 3A, an example interface 300A is illustrated and presented via example provider computing device 312, for viewing patient data, the patient data of which is utilized with a machine-learning platform to determine a recommendation of an implant (e.g. hardware component), one or more suggested bone grafting materials and/or procedures for a patient, in accordance with embodiments of the present disclosure. Interface 300A provides aspects of a patient data viewing tool 302A that allows a provider and/or a patient to view patient data. In some embodiments, the patient data of patient data viewing tool 302A is utilized via recommendation component 164 of FIG. 1 to determine a recommendation of implant, a bone graft material and/or procedure. FIG. 3B illustrates an example interface 300B for viewing patient data utilized to determine a recommendation of the optimal bone grafting materials and/or procedures for a patient, in accordance with embodiments of the present disclosure. In this example, interface 300B provides patient data viewing tool 302B that allows a provider and/or a patient to view imaging data 315 of the patient. In some embodiments, the patient data of patient data viewing tool 302B is utilized via recommendation component 164 of FIG. 1 to determine a recommendation of a bone graft material and/or procedure. The imaging here may be an X-ray to determine diagnosis, type of implant or biologic material including sizing and volumes of materials needed to incorporate in the procedure.

FIG. 3C illustrates an example interface (UI) 300C of a provider computing device (e.g., provider computing device 112 of FIG. 1) depicting a summation as to the patient, procedure, implants, etc. to trigger determining a recommendation of the optimal bone grafting materials and/or procedures for a patient, in accordance with embodiments of the present disclosure.

Aspects of this disclosure may incorporate an interface to engage and interact and/or enter inputs. In this example, interface 300C provides an action element 302C for the selection of the patient. Interface 300C provides an action element 304C for selection of the procedure. Interface 300C provides an action element 306C for the selection of specific materials, such as implants, for the procedure. Interface 300C provides an action element 308C for the selection of the available surgical instruments or devices for the procedure. Interface 300C provides an action element 310C for the selection of any preferences of the provider. In some implementations the various options available for selection are pre-determined and may be based on particular data about the patient, available resources from the provider, the surgeon's experience, cost, estimated recovery time, or other criteria. In some instances, there may be only one choice available.

FIG. 3D illustrates an example interface 300D of a provider computing device (e.g., provider computing device 112 of FIG. 1) for viewing a recommendation of the optimal bone grafting materials and/or procedures for a patient, in accordance with embodiments of the present disclosure. In this example, interface 300D provides a display element 302D for the recommendation of the optimal bone grafting materials and/or procedures for the patient. The display element 302D provides a two dimensional (2D) or three dimensional (3D) depiction to quickly visualize data to facilitate rapid decision making by a physician. For example, the visualization is configured to facilitate rapid decision making in less than 60 seconds. The graphical depiction and/or visualization can be designed and modified based on surgeon preferences. Thus, the graphical depiction and/or visualization can indicate performance-based outcomes of a particular bone graft material with the characteristic digital patient data. Interface 300D provides a display element 304D for the projected outcome of the recommendation. Interface 300D provides an action element 306D to provide surgical details of the recommendation. Interface 300D provides an action element 308D for pre-operation procedures of the recommendation. Interface 300D provides an action element 310D for post operation procedures of the recommendation. Interface 300D provides an action element 312D for the automated preparation of the bone graft material recommendation via bone graft material preparation hardware in communication with the application. Interface 300D provides an action element 314D for the automated preparation of the bone graft material recommendation and loading of the bone graft material on a robotic system via bone graft material preparation hardware and/or robotic system hardware in communication with the application.

In one aspect, product can be integrated in a marketing display to utilize the user interface as a shopping tool to compare various product, implants, and tools, among other devices.

FIG. 3E illustrates an example interface 300E presented via a provider computing device 312, for viewing the use of implants, bone grafting materials, instruments and/or procedures for a patient, in accordance with embodiments of the present disclosure. In this example, interface 300E provides a display element 302E characterizing implants, including materials and/or procedures for the patient as approved by the surgeon, medical staff, and anesthesia teams. Interface 300E provides a display element 304E for the recommended activities/behaviors and suggested alerts recommended by the provider to the patient. Interface 300E provides an action element 306E to provide patient data (e.g., examples of which are shown in FIGS. 3A and 3B). Interface 300E provides an action element 308E to provide medical provider or surgeon details. Embodiments of surgical details 308E comprise aspects of the surgery and may specify specific procedures, tools, staffing, and/or medicines such as anesthesia to be utilized in connection to the performance of the procedure by the surgeon.

Interface 300E provides an action element 310E for pre-operation procedures and recommendations suggested by the surgeon. Interface 300E provides an action element 312E for post operation procedures and behaviors suggested to patient by the surgeon. Interface 300E provides an action element 314E for any alerts regarding the recommendation approved by the surgeon.

Turning now to FIG. 9, an example patient user interface 900A presented via a patient computing device 916A, is illustratively provided. Embodiments of example interface 900A are configured for use by and presentation to a particular patient. For example, interface 900A is configured for viewing the aspects of implants, bone grafting materials, instruments and/or procedures for a patient, in accordance with embodiments described herein. In this example, interface 900A provides a display element 902A characterizing implants, including materials, pharmaceutical applications/drugs, and/or procedures for the patient as approved by the surgeon, medical staff, and anesthesia teams. Interface 900A provides a display element 904A for the recommended activities/behaviors 903A and alerts 905A recommended by the provider to the patient; the activities 903A and alerts 905A are generated by the provider at the provider user interface 300E at FIG. 3E. Interface 900A provides an action element 906A to provide patient data (e.g., examples of which are shown in FIGS. 3A and 3B). Interface 900A provides an action element 908A to provide medical provider or surgeon details. Embodiments of surgical details 908A comprise aspects of the surgery and may specify specific procedures, tools, staffing, and/or medicines such as anesthesia to be utilized in connection to the performance of the procedure by the surgeon.

Interface 900A provides an action element 910A for pre-operation procedures and recommendations suggested by the surgeon. Interface 900A provides an action element 912A for post operation procedures and behaviors suggested to patient by the surgeon. Interface 900A provides an action element 914A for any alerts regarding the recommendation approved by the surgeon. The patient user interface is provided to the patient from the provider to support customer needs and better serve the patient. Wearable devices by the patient may also integrate here to further populate patient data (and cross-populate the provider database) to keep provider updated as to patient condition, status and behaviors. The system here may also notify the patient, the notifications suggested by provider and approved by the patient, to create an interactive workflow between the users.

### Example Process Flows

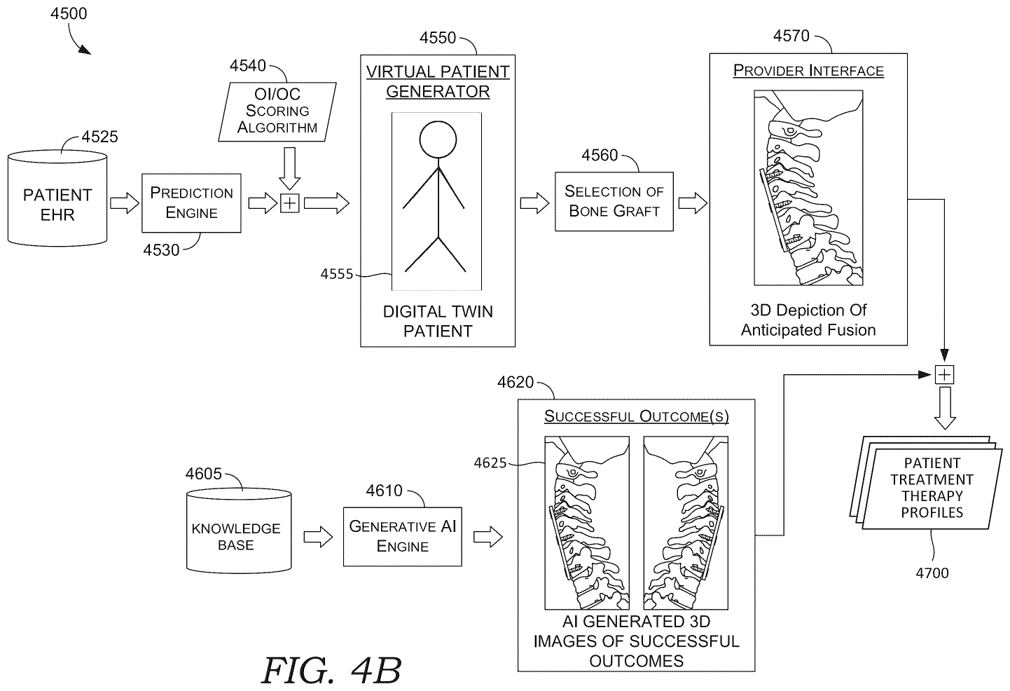

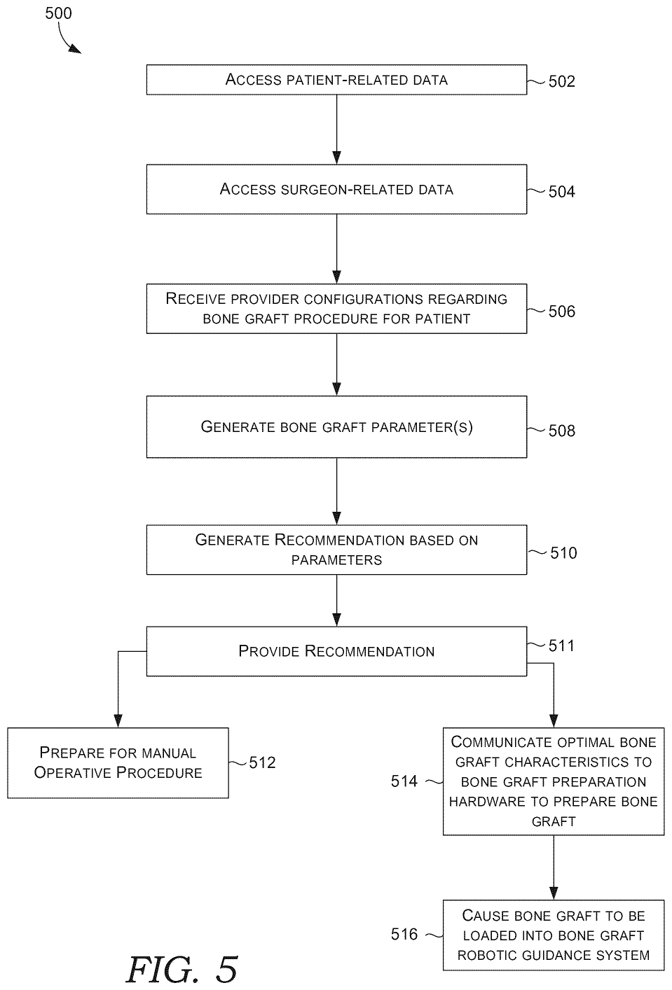

With reference now to FIGS. 4A, 4B and 5, flow diagrams are provided illustrating various methods. Each block of the methods 400, 4500, and 500, as well as other computer-implemented methods described herein comprise a computing process performed using any combination of hardware, firmware, and/or software. For instance, in some embodiments, various functions are carried out by a processor executing instructions stored in memory. In some cases, the methods are embodied as computer-usable instructions stored on computer storage media. In some implementations, the methods are provided by a standalone application, a service or hosted service (standalone or in combination with another hosted service), or a plug-in to another product, to name a few.

FIG. 4A is a flow diagram showing a method 400 for determining a recommendation of spinal implant, the optimal bone grafting materials and/or procedures for a patient, in accordance with embodiments of the present disclosure. Initially, a digital patient 401 includes patient data at block, patient data 402 (e.g., EMR/EHR 132 of FIG. 1) is accessed. For example, the patient's data can include any data regarding the patient, including, but not limited to, imaging 405 for the patient, such as thermal imaging, CT scans, MRI scans, X-RAYs, PET scan, ultrasound, and/or etc. Generally, the patient's data 402 includes, but is not limited to, EHRs can include any health data for the patient, such as an ECG or EKG, labs/pathology, wearable technology, such as sensors, biometrics, biomarkers, OI, bone health, calcium compositions, images/video/audio of the patient, data from genomics tools, such as a third party database, diagnostic records, blood testing, histology, blood pressure, heart rate, recurring status, compliance with medicines and prescribed treatment, activities, fusion rates, pain thresholds and assessments, age, weight, height, other biometrics, bone health, calcium compositions, personal demographic data, such as age, sex, and/or economic status, insurance coverage and/or payouts, diagnostics, social conditions, psychological conditions, physical conditions or stress conditions, geographical location, bone health, blood work, blood pressure, pain, and health monitoring information, and any other information regarding the patient. Images are translated to digital configurations at an automatic labeling and geometric configuration at step 410.

At block 420, data regarding a surgeon performing the procedure is accessed at surgeon data model 421. System physiology 422 corresponds to the patient specific needs as to diagnosis and/or treatment/therapy/procedure, and further comprising osteogenic capacity score. Implant characterization including OI, OC, and OG for the implant, physics-based modeling of a product, biochemical attributes, physical characteristics, surface and interactive chemistries, and biocompatibility are integrated too. The data regarding the surgeon can include any data of the surgeon as determined by the neural network data correlations, and including preferences previously selected and configured to the preferred outcomes of prior surgical procedures (e.g., procedures involving bone grafts, procedures similar to the procedure selected for the patient, and/or etc.), time to perform the prior procedures, data regarding learning experiences regarding surgical procedures, and/or the like. In some embodiments, the data regarding the surgeon may include data regarding a group of surgeons (e.g., a group at a particular hospital or in a particular healthcare network).

At block 430, configurations are received from the provider regarding the procedure for the patient involving the bone graft. In some embodiments, the provider (e.g., a surgeon, doctor, nurse, counselor, health insurance administrator, and/or medical specialist, such as a medical materials or device manufacturer, supplier, sales representative, or medical consultant for bone graft materials, tools, or aspects thereof) determines a representation of a procedure involving a bone graft. For example, a surgeon selects a location of a disc removal for a patient that would require a bone graft. In some embodiments, the surgeon determines or selects the various characteristics of the procedure, such as the type of implants (e.g., a titanium, carbon fiber, or a bio-compatible polymer cage, fixation devices, and any other device implanted during a surgical surgery that utilizes a bone graft), they type of bone graft materials available for the procedure (e.g., whether ICBG is available for the patient, access to specific autografts, allografts, or synthetic bone grafts), instruments available for the procedure (e.g., such as whether the surgeon has access to a robotic system for assisting with the bone graft procedure) and/or any preferences of the surgeon (e.g., such as particular grafting materials that the surgeon prefers). In some implementations, the data regarding the user's selections can include data regarding the user's preferences as selected by the user via an application. For example, a surgeon may select specific preferences for specific materials and/or procedures. As another example, a medical specialist, such as a medical materials or device manufacturer, supplier, sales representative, or medical consultant selects a specific preference for particular product or group of products, such as a particular allograft or DBM product, CBM or VBM product, or synthetic graft.

At block 440, bone graft parameters are determined by predictive engine 445 using medical knowledge through literature, clinical studies, clinical procedure, or otherwise may be integrated into the neural network to determine prior use of implants corresponding to public reviews at block 450. Osteogenic capacity of a patient may be determined through a scoring mechanism or range 0-4, 0 least likely to grow bone to 4 most likely to grow bone. In some embodiments, optimal bone graft parameters are generated via a neural network. The bone graft parameters are determined to be optimized to maximize a likelihood of a successful clinical outcome, such as a likelihood of fusion success, reduced pain, improved disability and function. Alternatively or in addition, bone graft parameters are determined to be optimized for other criteria such as to minimize patient invasiveness, reduce costs, or utilize available resources, which may include materials, tools, and surgeon availability.

For example and as further described herein, in one embodiment, a neural network is trained to determine one or more bone graft parameters such as materials, a care plan or procedure, or aspect thereof, that optimizes the ratio of bone graft characteristics (e.g., osteoconduction, osteoinduction, and osteogenesis) of the implanted construct to maximize clinical outcome and/or minimize cost via historical data regarding bone graft procedures. The optimal parameter may be provided as a clinical decision support recommendation to a caregiver or provider, such as a surgeon performing the procedure. In some embodiments, the trained neural network utilizes the patient's EHR(s), historical surgical data regarding a surgeon who may be designated to perform the bone graft procedure, and/or other data regarding a user's selection to determine the recommendation of the optimal bone grafting material and/or procedure for the patient. In some embodiments, a bone grafting parameter is determined by the trained neural network, such as a bone grafting material or procedure, is provided along with supplemental content, such as a recommendation, instructional or procedural information, clinical data, or data supporting or explaining the neural network output. For example, the trained neural network can recommend an optimal implant, such as a titanium, carbon fiber, or a bio-compatible polymer cage for an interbody fusion or an extradiscal fixation device, for the procedure and/or bone grafting materials.

At block 450, patient specific osteogenic capacity scores (vs. biologic osteogenesis (OG)) are incorporated with the function of one or more optimal bone graft parameters determined at block 440 are provided to the user. Medical knowledge of 445 may also be integrated to generate a virtual treatment protocol to include: procedure, implant products, or other biologics, implants or instruments. For example, the parameter(s) may be displayed on a clinician user interface (such as example interface 300C of FIG. 3C). Additionally, or alternatively, the parameters may be stored, and/or may be used for further determining a patient care plan, procedural instructions, or treatment for the patient. For example, the recommendation of the optimal bone grafting material and/or procedure for the patient is output to the device of the provider (e.g., the surgeon) for display and/or stored. In some embodiments, the data output regarding the recommendation of the optimal bone grafting material and/or procedure for the patient can include data regarding the optimal bone grafting material (e.g., volume, texture, hydration, material, bone growth factor, such as BMP2, etc.), the optimal surgical method of insertion, the projected clinical outcomes, regulatory compliance data, pre-operation planning (e.g., pre-habilitation), post-operative care (e.g., rehabilitation, follow-up procedures, pain medication, steroids, medications, etc.), surgery details (e.g., procedure type, time, anesthesia, etc.), regulatory compliance data, and any other data regarding the materials or procedure. In some embodiments, data regarding the recommendation of the bone grafting material and/or procedure is output to an application on the device of the patient upon approval by the provider (e.g., the surgeon). For example, the output data can include pre-operation planning, surgery details, post-surgical review, follow-up procedures, etc. In some embodiments, the provider can utilize the recommendation to manually prepare the bone grafting material in a device, such as a syringe, for injection of the bone graft into the patient during the procedure.

Device knowledge 455 that is integrated through wearable sensors, telemetry, or other monitoring software and/or devices is then optimized at step 460 to determine the best treatment parameters (i.e. best determined by preferred clinical outcomes) for a patient (or digital patient) at a virtual treatment protocol 470. Prediction engine 472 further may integrate a feedback loop that allows patient outcomes 471 to solicit additional inputs 480 to further optimize treatment, surgical planning, and product selection for implant. The models of FIG. 4A can be characterized as a digital twin or an avatar of the patient.

FIG. 4B is a flow diagram describing the generation of a patient digital twin and how that is combined with a generative AI model for an optimized patient therapy. 4525 is an EHR database containing patient database such as but not limited to ECG or EKG, labs/pathology, wearable technology, such as sensors, biometrics, biomarkers, OI, bone health, calcium compositions, images/video/audio of the patient, data from genomics tools, such as a third party database, diagnostic records, blood testing, histology, blood pressure, heart rate, recurring status, compliance with medicines and prescribed treatment, activities, fusion rates, pain thresholds and assessments, age, weight, height, other biometrics, bone health, calcium compositions, personal demographic data, such as age, sex, and/or economic status, insurance coverage and/or payouts, diagnostics, social conditions, psychological conditions, physical conditions or stress conditions, geographical location, bone health, blood work, blood pressure, pain, and health monitoring information, and any other information regarding the patient. 4540 is the prediction engine algorithm combines the data either with physics or data based model to create a digital twin. 4550 is the digital twin can be understood as a calibrated physical model or a trained data model, and it's virtually indistinguishable from the physical entity it's representing for the question being asked. The digital twin is afterwards used by 4560, the selection of bone graft that by testing multiple options in the digital twin, selects the treatment with the highest changes of success. 4570 is the user interface that show the results to the health care provider. 4650 is a non-patient-specific database of information containing research or bibliographic data regarding optimal bone graft for each case. 4610 is a generative AI model that would extract the optimal treatment for that patient based on the knowledge database and obtaining (4620) the optimal result. That information combined with the digital twin prediction provides the optimal patient treatment therapy profiles.

For exemplary purposes, and not limitation, genetic polymorphisms have been associated with disc degeneration. These polymorphisms in genes result in microscopic and macroscopic changes that are characteristic of degeneration progression. Biochemical markers related to bone turnover, therefore, could then be integrated into training artificial intelligence (AI) coding and modeling to assess a patient's osteogenic capacity. Overall bone health, however, and correlations with a particular bone grafting product, or other biologic, may be integrated into the model to provide an optimal patient outcome. Some of these biomarkers include:
Bone Formation Biomarkers
Serum total alkaline phosphatase (ALP)
Serum osteocalcin (OC)
Serum procollagen type 1 N-terminal propeptide (P1NP), Serum procollagen type 1 C-terminal propeptide (P1CP)
Bone Resorption Biomarkers
Urinary hydroxyproline (HYP)
Urinary free deoxypyridinoline (DPD)
Urinary total pyridinoline (PYD)
Urinary collagen type 1 cross-linked N-telopeptide (NTX-1)
Urinary or serum collagen type 1 cross-linked C-telopeptide (CTX-1)
Bone sialoprotein (BSP)
Tartrate-resistant acid phosphatase 5b (TRAP 5b)
   In an AI model, the degradation or loss of collagen by a marker COL1 or COL9, COL11, C1LP, ASPN, or GDF5, for example, would indicate a loss of structural integrity of the bone. In another aspect, the degradation or loss of proteoglycan, as indicated by VDR, HAPLN, CLP, FN, ASPN, could be indicative of a loss of hydrostatic pressure. Another aspect uses increased levels of ILl, IL6, or COX2 to indicate increased inflammation and therefore greater symptoms of pain. MMPs of MMP1, MMP2, MMP3, T1MP, R1, and R6 can also be indicative here of disc/bone degeneration. Such biomarkers have input selections within an AI model to determine patient health, bone health, or act as a factor indicative of biologic selection, or any implant selection. Furthermore, biomarkers can be early markers for risk assessment, used as diagnostic markers and routine clinical care, interoperative markers for determining surgical or treatment protocols, or postoperative markers for predictive outcomes. The marker is thus integrated in the algorithm and trained learning model to appropriately designate the outcome selection desired.

FIG. 5 is a flow diagram showing a method 500 for determining a recommendation of the optimal bone grafting materials and/or procedures determined for a particular patient, in accordance with embodiments of the present disclosure. Initially, at block 502, patient-related data (e.g., EMR/EHR 132 of FIG. 1) is accessed. For example, the patient's data can include any data regarding the patient, including, but not limited to, imaging for the patient, such as thermal imaging, CT scans, MRI scans, X-RAYs, PET scan, ultrasound, and/or etc. Generally, the patient's EHRs can include any health data for the patient, such as an ECG or EKG, labs/pathology, wearable technology, such as sensors, biometrics, OI, bone health, calcium compositions, images/video/audio of the patient, data from genomics tools, such as a third party database, diagnostic records, blood testing, histology, blood pressure, heart rate, recurring status, compliance with medicines and prescribed treatment, activities, fusion rates, pain thresholds and assessments, age, weight, height, other biometrics, bone health, calcium compositions, personal demographic data, such as age, sex, and/or economic status, insurance coverage and/or payouts, diagnostics, social conditions, psychological conditions, physical conditions or stress conditions, geographical location, bone health, blood work, blood pressure, pain, and health monitoring information, and any other information regarding the patient. In some embodiments, patient-related data includes data regarding other patients similar to a particular patient, such as patients that share a condition with, have received treatment or undergone a procedure similar to a procedure or treatment considered for a particular patient. In some embodiments of block 502, the patient-related data is accessed via input data accessing component 162, or more generally by server 150 or other computing device described in FIG. 1.

At block 504, data regarding the surgeon expected to perform the procedure (e.g., provider data 134 of FIG. 1) is accessed. Data regarding the surgeon can include any data associated with a surgeon expected to be involved in performing a procedure or treatment on the patient. Without limitation, such data can include demographic data; location data; specialty; education; experience; historical data regarding prior procedures performed by the surgeon, which may include data regarding the outcomes; admissions to hospitals; licenses; certifications; preferences of the surgeon for particular product, or product selection based on prior experiences, clinical use, and selections byte surgeon for particular procedures, implants, and biologics. The outcomes of prior surgical procedures, such as procedures involving bone grafts, procedures similar to the procedure selected for the patient, and/or the like, time taken to perform the prior procedures, data regarding learning experiences regarding surgical procedures, and/or the like further feeds into the platform to facilitate decision making as driven by data analytics at the processor and delivered as outcomes at the UI. In some embodiments, the data regarding the surgeon may include data regarding a group of surgeons (e.g., a group at a particular hospital or in a particular healthcare network). Some embodiments of block 504 are carried out by via input data accessing component 162, or more generally by server 150 or other computing device described in FIG. 1.

At block 506, selections are received from the provider (e.g., as input via provider interface 120 and accessed via input data accessing component 162 of FIG. 1) regarding the procedure for the patient involving the bone graft. In some embodiments, the provider selects or otherwise determines a representation of a procedure involving a bone graft during pre-operative planning. For robotic surgery, this preoperative planning representation may be configured to integrate or cross-populate across an application programming interface (API) of a hub that indirectly connects with the medical device, or to directly connect with the device. For example, a surgeon selects a location of a disc removal for a patient that would require a bone graft. In some embodiments, the surgeon selects the various characteristics of the procedure, such as the type of implants (e.g., a titanium, carbon fiber, or a bio-compatible polymer cage, fixation devices, and any other device implanted during a surgical surgery that utilizes a bone graft), the type of bone graft materials available for the procedure (e.g., whether ICBG is available for the patient, access to specific autografts, allografts, or synthetic bone grafts), instruments available for the procedure (e.g., such as whether the surgeon has access to a robotic system for assisting with the bone graft procedure) and/or any preferences of the surgeon (e.g., such as particular grafting materials that the surgeon prefers). In some implementations, the data regarding the user's selections can include data regarding the user's preferences as selected by the user via an application. For example, a surgeon may select specific preferences for specific materials and/or procedures. As another example, a sales representative may select a specific preference for particular product or group of products, such as a particular allograft or DBM product, CBM or VBM product, or synthetic graft, including any and all biologics.

At block 508, optimal bone graft parameters are generated. In particular, embodiments of block 508 generate or otherwise determine the bone graft parameters based on the selections of block 506. Some embodiments of block 508 comprise determining or configuring bone graft characteristic that correlate with parameters of the digital patient in order to maximize the clinical outcome. In some embodiments, bone graft parameter logic is utilized to generate or configure the one or more bone graft parameters accordingly, such as implant/bone graft parameter logic 135 of FIG. 1 or optimal bone graft materials and/or procedure recommendation logic 206, described in FIG. 2. Some embodiments of block 508 are performed by implant and/or bone graft optimization component 160 of FIG. 1.

At block 510, a recommendation is generated. Embodiments of block 510 generate a recommendation based on one or more of the bone graft parameters determined at block 508. In an embodiment, the recommendation is generated based on optimal bone graft characteristics that are correlated with parameters of a digital patient and are configured to maximize the clinical outcome. Some embodiments of block 510 utilize recommendation logic 137 or FIG. 1, which may include application of an artificial neural network, as described further in connection with FIG. 1. For example and as described further in connection with FIG. 1, in one implementation, a machine learning system, such as a neural network, of recommendation logic 137 is trained to determine a recommendation of a bone graft material and/or procedure that optimizes the bone graft characteristics (e.g., osteoconduction, osteoinduction, and osteogenesis) of the implanted construct to maximize clinical outcome and/or minimize cost via historical data regarding bone graft procedures. Accordingly, at block 510, the trained machine learning model (recommendation logic 137, such as a trained neural network) is utilized by recommendation component 164 (or other computing device) of FIG. 1, to input aspects of the patient's EHRs, surgeon-related data such as historical surgical data regarding the surgeon designated to perform the bone graft procedure, data regarding the provider's selections, and/or aspects of other data accessed or determined at block 502, 504, or 506. In this way, the trained neural network is applied, using the input data, to determine the recommendation of the optimal bone grafting material and/or procedure for the particular patient. In some embodiments, the trained neural network determines a recommendation of the optimal bone grafting material, along with recommendations regarding the optimal procedure. For example, the trained neural network can recommend an optimal implant, such as a titanium, carbon fiber, or a bio-compatible polymer cage for an interbody fusion or an extradiscal fixation device, for the procedure and/or bone grafting materials. Some embodiments of block 510 are carried out by recommendation component 164 of FIG. 1.

At block 511, the recommendation is provided. Some embodiments of block 511 comprise presenting an indication of the recommendation via a computer-user interface, such as provider interface 120 of a provider computing device 112, described in connection to FIG. 1, for example, at block 511, the recommendation, determined at block 510, is provided to a provider, such as a surgeon involved in performing a procedure on the patient. In some embodiments, the recommendation is provided as part of a computing application or service operating on a provider computing device, such as implant optimization tool 124 of FIG. 1. In some embodiments, and as further described herein, the recommendation further includes details supporting the recommendation or includes a hyperlink for accessing information used to generate the recommendation and/or supporting the recommendation. In some embodiments, a user-interface element is presented with the recommendation to receive input from the provider (e.g. the surgeon) to accept or disregard the recommendation. In this way, by determining and presenting the recommendation, certain embodiments of the technology described herein operate as a clinical decision support platform to generate and provide decision support for a human healthcare provider, such as the surgeon performing a procedure on the patient. The clinical decision support platform may be configured to allow for the provider, such as the surgeon performing a procedure on the patient, to make the decision as to whether to accept or dismiss the recommendation. For example, via the user-interface element presented with the recommendation described above. In some embodiments, the provider's decision to accept or reject a recommendation is recorded for use in the generation of future recommendations or for further training the machine learning system.

In some embodiments of block 511, subsequent or concurrent with providing the recommendation and receiving acceptance or confirmation of the recommendation, input is received from the provider (e.g., the surgeon) as to whether the recommendation is to be carried out manually or using automated/machine assistance, such as by using robotic assistance from a robotic system 176, as described in FIG. 1. For example, some embodiments of block 511 further comprise providing, via provider interface 120, a graphical user-interface element such as a button or audio prompt (or other UI means) for the soliciting input from the provider as to whether an accepted recommendation is to be carried out manually or using automated/machine assistance.

In other embodiments, a determination at block 511 as to whether an accepted recommendation is to be carried out manually or using automated/machine assistance is pre-determined. For example, the determination regarding whether certain recommendations are carried out manually or using automated/machine assistance is based on the provider's preferences, such as predetermined preferences specified by the provider or based past decisions involving similar recommendations, or is based on a healthcare administrator-configured setting, such as a setting specified by a hospital system. The pre-determined setting may include an indication as to whether all procedures, specific procedures, or specific types of procedures (or other aspects of a recommendation) are to be carried out manually or using automated/machine assistance. These predetermined settings may be as configuration settings associated with provider computing device 112, an account associated with the provider, or a configuration file associated with the healthcare facility or hospital system. Accordingly in these embodiments of block 511, it is not necessary to receive input from the provider as to whether an accepted recommendation is to be carried out manually or using automated/machine assistance. Nevertheless, some implementations where a predetermined setting us used including functionality for providing an indication that an accepted recommendation will be carried out manually or automated/machine assistance. For example, upon accepting a recommendation, a surgeon performing the procedure on a patient is presented with an indication that the accepted recommendation is performed using automated/machine assistance.

Based on a determination at block 511 that the accepted recommendation comprises a manually operative procedure, method 500 proceeds to block 512. On the other hand, based on a determination at block 511 that the accepted recommendation will be carried out using automated/machine assistance, then method 500 proceeds to block 514. Accordingly, at block 512, a manual operative procedure will be utilized. Some embodiments of method 500 terminate after a determination is made that the recommendation will utilize a manual operative procedure. In other embodiments, information regarding the manual operative procedure is communicates to the provider or other individuals involved in treating the patient including the patient in some instances. For example, instructions for performing the recommended procedure, such as the application of a particular bone graft on the patient, are presented to the provider via provider interface 120. In some instances, computer instructions are also communicated, such as instructions to facilitate provisioning equipment, materials, or staff.

Method 500 proceeds to block 514 where it is determined that an accepted recommendation uses automated/machine assistance. Accordingly, at block 514, parameters regarding the accepted recommendation, which may include one or more of the parameters determined at step 508, are communicated to facilitate the preparation of the bone graft or implant material using automated or machine assistance. For example, in an embodiment, the optimal bone graft characteristics of the bone graft material are communicated (via bone graft hardware communication component 166 of FIG. 1) to bone graft preparation hardware (e.g., bone graft implant hardware 172 of FIG. 1) In some embodiments instance, the recommendation of the optimal bone grafting material and/or procedure for the patient is output to the device of the provider (e.g., the surgeon) for display and/or stored. The provider (e.g., the surgeon) can thus approve or review and approve aspects of the recommendation. In some embodiments, the provider (e.g., the surgeon) can select the recommendation in the computing application running on the provider computing device, in order to cause bone graft material preparation hardware in communication with the application to automatically prepare the bone grafting material or to automatically issue instructions or provision resources to facilitate the preparation of the material.

At block 516, bone graft material is caused to be automatically loaded into a robotic guidance system, such as robotic system 176 of FIG. 1. In some embodiments of block 516, subsequent to the provider accepting or confirming the recommendation, bone graft material corresponding to the accepted recommendation is caused to be loaded into a robotic guidance system. Some embodiments of block 516 are carried out via implant optimization tool 124 of provider interface 120 of FIG. 1. In particular, aspects of the recommendation corresponding to the bone graft material, such as one or more bone graft parameters, is communicated (e.g., via bone graft hardware communication component 166 of FIG. 1) to programmatically cause the bone graft material to be loaded into the robotic system (e.g., robotic system 176 of FIG. 1). In this way, the provider is assisted automatically or semi-automatically in performing aspects of the bone graft procedure. In some embodiments, after the bone graft material is prepared by the bone graft material preparation hardware, a surgeon can select an action element in the application to cause the bone graft preparation hardware to load the prepared bone graft material onto a robotic system, such as a robotic arm system, to assist the surgeon in performing the bone graft procedure.

FIG. 8 is a representative decision support system 800 integrated to utilize ML/DL and generative AI to display real-time visualizations of data that correlate a patient with a suggested surgical implant, bone graft, biologic, tools and/or procedure, in accordance with embodiments herein. Provider/sales representative 802 select data, such as data from EMR 804, spine patient records 806, surgical records 808, and imaging 810, are input into request to populate patient profile 814. The patient profile can be provided to a patient via a patient UI 812. The patient record is automatically or manually generated at block 816. The patient record is created and the corresponding data is stored at block 818. Machine learning, deep learning, and or AI logic 819 is applied to the patient record to generate a visualization at block 820, such as a two-dimensional or three-dimensional depictions created across various screens that can include data, including, but not limited to EMR, pathology, bloodwork, imaging, patient records (such as through a patient UI), etc.). At block 821, various data can be determined from the visualization and displayed via various screens of a UI. For example, at block 822, data can be extracted and/or depicted in charts and/or tables. At block 824, the implant recommendation based on the procedure for the patient can be determined. At block 826, the surgical protocol can be determined, such as surgeon, implants to be used, materials/instrument, bone graft, drugs, reactions, etc. At block 828, the predicted outcome with various bone graft materials. At block 830, recommended alerts, pre-operative care, post-operative care, suggestions, selected to push to EMR and/or patient's UI. This data of block 821 can be fed back into patient record of block 816. The provider and/or sales representative makes a selection at block 832. The selected implants, bone graft materials, drugs, etc. of block 833 can be displayed on the provider UI at block 850. With respect to block 821, at block 852, each of the screens can have selectable buttons to extrapolate detail as to patient data, instrument data (e.g., make, model number, part number, etc.), implants, bone graft, biologic, regulatory guidance notices, etc. At block 858, hospital, insurance and/or administrators can determine access, limitations, and cost at block 854 and reimbursement rates at block 856 to determine whether the procedure can be covered or how supported within hospital administration. At block 860, there can be real-time access feedback for the entire system. For exemplary purposes, and not limitation, the system models operate and update in real-time and/or point of time snapshot configurations.

### Example Computing Environments

Having described various implementations, several example computing environments suitable for implementing embodiments of the disclosure are now described, including an example computing device and an example distributed computing environment in FIGS. 6 and 7, respectively.

Aspects of this disclosure may be described in the general context of computer code or machine-useable instructions, including computer-useable or computer-executable instructions, such as program modules, being executed by a computer or other machine such as a smartphone, a tablet PC, or other mobile device, server, or client device, such as a digital medical device. Generally, program modules, including routines, programs, objects, components, data structures, and the like, refer to code that performs particular tasks or implements particular abstract data types. Embodiments of this disclosure may be practiced in a variety of system configurations, including mobile devices, consumer or medical electronics, general-purpose computers, more specialty computing devices, or the like. Embodiments of the disclosure may also be practiced in distributed computing environments where tasks are performed by remote-processing devices that are linked through a communications network. In a distributed computing environment, program modules may be located in both local and remote computer storage media including memory storage devices.

Some embodiments comprise an end-to-end software-based system that can operate within system components described herein to operate computer hardware to provide system functionality. At a low level, hardware processors may execute instructions selected from a machine language (also referred to as machine code or native) instruction set for a given processor. The processor recognizes the native instructions and performs corresponding low level functions relating to, for example, logic, control, and memory operations. Low level software written in machine code can provide more complex functionality to higher levels of software. Accordingly, in some embodiments, computer-executable instructions may include any software, including low level software written in machine code, higher level software, such as application software, and any combination thereof. In this regard, the system components can manage system resources and provide services for system functionality. Any other variations and combinations thereof are contemplated with embodiments of the present disclosure.

Referring to the drawings generally and initially to FIG. 6 in particular, an example computing device is provided and referred to generally as computing device 600. The computing device 600 is but one example of a suitable computing environment and is not intended to suggest any limitation as to the scope of use or functionality of embodiments of the disclosure. Neither should the computing device 600 be interpreted as having any dependency or requirement relating to any one or combination of components illustrated.

As shown in FIG. 6, computing device 600 includes a bus 610 that directly or indirectly couples the following devices: memory 612, one or more processors 614, one or more presentation components 616, input/output (I/O) ports 618, one or more I/O components 620, an illustrative power supply 622, and a radio(s) 624. Bus 610 represents what may be one or more busses (such as an address bus, data bus, or combination thereof). Although the various blocks of FIG. 6 are shown with lines for the sake of clarity, these blocks represent logical, not necessarily actual, components. For example, a presentation component such as a display device may be an I/O component. Also, processors have memory. Accordingly, it is recognized that such is the nature of the art and thus the diagram of FIG. 6 is merely illustrative of an example computing device that can be used in connection with one or more embodiments of the present disclosure. Distinction is not made between such categories as “workstation,” “server,” “laptop,” or “handheld device,” as all are contemplated within the scope of FIG. 6 and with reference to “computing device.”

Computing device 600 typically includes or uses a variety of computer-readable media. Computer-readable media can be any available media that can be accessed by computing device 600 and includes both volatile and nonvolatile, removable and non-removable media. By way of example, computer-readable media may comprise computer storage media and communication media. Computer storage media includes both volatile and nonvolatile, removable and non-removable media implemented in any method or technology for storage of information such as computer-readable instructions, data structures, program modules, or other data. Computer storage media includes random access memory (RAM), read only memory (ROM), electrically erasable programmable read-only memory (EEPROM), flash memory or other memory technology, CD-ROM, digital versatile disks (DVDs) or other optical disk storage, magnetic cassettes, magnetic tape, magnetic disk storage or other magnetic storage devices, or any other hardware medium which can be used to store the desired information and which can be accessed by computing device 600. Computer storage media does not comprise a propagated data signal. Communication media typically embodies computer-readable instructions, data structures, program modules, or other data in a modulated data signal such as a carrier wave or other transport mechanism and includes any information delivery media. The term “modulated data signal” means a signal that has one or more of its characteristics set or changed in such a manner so as to encode information in the signal. By way of example, communication media includes wire-transmitted media, such as a wired network or direct-wired connection, and wireless-transmitted media, such as acoustic, radio frequency (RF), infrared, and other wireless media. Combinations of any of the above should also be included within the scope of computer-readable media.

Memory 612 includes computer storage media in the form of volatile and/or nonvolatile memory. The memory may be removable, non-removable, or a combination thereof. Example hardware devices include, for example, solid-state memory, hard drives, and optical-disc drives. Computing device 600 includes one or more processors 614 that read data from various entities such as memory 612 or I/O components 620. As used herein, the term processor or “a processer” may refer to more than one computer processor. For example, the term processor (or “a processor”) may refer to at least one processor, which may be a physical or virtual processor, such as a computer processor on a virtual machine. The term processor (or “a processor”) also may refer to a plurality of processors, each of which may be physical or virtual, such as a multiprocessor system, distributed processing or distributed computing architecture, cloud computing system, or parallel processing by more than a single processor. Further, various operations described herein as being executed or performed by a processor may be performed by more than one processor.

Presentation component(s) 616 present data indications to a user or other device. Example presentation components include a display device or screen, speaker, printing component, vibrating component, and the like. The I/O ports 618 allow computing device 600 to be logically coupled to other devices, including I/O components 620, some of which may be built in. Illustrative components include a keyboard, buttons, touch screen or touch-sensitive surface, microphone, camera, mouse, joystick, control pad, satellite dish, scanner, printer, or a wireless peripheral device. The I/O components 620 may provide a natural user interface (NUI) (such as touch interaction, pen (or stylus) gesture, and gaze detection), and the like, that processes air gestures, voice, or other physiological inputs generated by a user. In some instances, inputs may be transmitted to an appropriate network element for further processing. An NUI may implement any combination of speech recognition, touch and stylus recognition, facial recognition, biometric recognition, gesture recognition both on screen and adjacent to the screen, air gestures, head and eye tracking, and touch recognition associated with displays on the computing device 600. The computing device 600 may be equipped with depth cameras, such as stereoscopic camera systems, infrared camera systems, RGB camera systems, and combinations of these, for gesture detection and recognition. Additionally, the computing device 600 may be equipped with accelerometers or gyroscopes that enable detection of motion. The output of the accelerometers or gyroscopes may be provided to the display of the computing device 600 to render immersive augmented reality or virtual reality. The connection between the I/O components 620 and processor(s) 614 may be direct or via a coupling utilizing a serial port, parallel port, and/or other interface and/or system bus known in the art.

Some embodiments of computing device 600 include one or more radio(s) 624 (or similar wireless communication components). The radio transmits and receives radio or wireless communications. The computing device 600 may comprise a wireless terminal adapted to receive communications and media over various wireless networks. Computing device 600 may communicate via wireless protocols, such as code division multiple access (“CDMA”), global system for mobiles (“GSM”), or time division multiple access (“TDMA”), as well as others, to communicate with other devices. The radio communications may be a short-range connection, a long-range connection, or a combination of both a short-range and a long-range wireless telecommunications connection. Short-range and long-range types of connections do not refer to the spatial relation between two devices, but instead refers to short range and long range as different categories, or types, of connections (for example, a primary connection and a secondary connection). A short-range connection may include, by way of example, a Wi-Fi® connection to a device (e.g., mobile hotspot) that provides access to a wireless communications network, such as a WLAN connection using the 802.11 protocol; a Bluetooth connection to another computing device is a second example of a short-range connection, or a near-field communication connection. A long-range connection may include a connection using, by way of example, one or more of CDMA, General Packet Radio Service (GPRS), GSM, TDMA, and 802.16 protocols, or other long-range communication protocols used by mobile devices.

Referring now to FIG. 7, an example distributed computing environment 700 is illustratively provided, in which implementations of the present disclosure may be employed. In particular, FIG. 7 shows a high-level architecture of an example cloud computing platform 710 that can host a technical solution environment, or a portion thereof (e.g., a data trustee environment). It should be understood that this and other arrangements described herein are set forth only as examples. For example, as described above, many of the elements described herein may be implemented as discrete or distributed components or in conjunction with other components, and in any suitable combination and location. Other arrangements and elements (e.g., machines, interfaces, functions, orders, and groupings of functions) can be used in addition to or instead of those shown.

Data centers can support a distributed computing environment 700 that includes a cloud computing platform 710, a rack 720, and a node 730 (e.g., computing devices, processing units, or blades) in rack 720. The technical solution environment can be implemented with cloud computing platform 710, which runs cloud services, such as cloud computing applications, across different data centers and geographic regions. Cloud computing platform 710 can implement fabric controller 740 component for provisioning and managing resource allocation, deployment, upgrade, and management of cloud services. Typically, cloud computing platform 710 acts to store data or run service applications in a distributed manner. Cloud computing platform 710 in a data center can be configured to host and support operation of endpoints of a particular service. Cloud computing platform 710 may be a public cloud, a private cloud, or a dedicated cloud.

Node 730 can be provisioned with host 750 (e.g., operating system or runtime environment) running a defined software stack on node 730. Node 730 can also be configured to perform specialized functionality (e.g., compute nodes or storage nodes) within cloud computing platform 710. Node 730 is allocated to run one or more portions of a service application of a tenant. A tenant can refer to a customer utilizing resources of cloud computing platform 710. Service application components of cloud computing platform 710 that support a particular tenant can be referred to as a multi-tenant infrastructure or tenancy. A cloud service may comprise any software, or portions of software, that run on top of, or access storage and computing device locations within, a datacenter.

When more than one separate service application is being supported by nodes 730, nodes 730 may be partitioned into virtual machines (e.g., virtual machine 752 and virtual machine 754). Physical machines can also concurrently run separate service applications. The virtual machines or physical machines can be configured as individualized computing environments that are supported by resources 760 (such as computer resources, which may comprise hardware resources and/or software resources) in cloud computing platform 710. It is contemplated that resources 760 can be configured for specific service applications. Further, each service application may be divided into functional portions such that each functional portion is able to run on a separate virtual machine. In cloud computing platform 710, multiple servers may be used to run service applications and perform data storage operations in a cluster. In particular, the servers may perform data operations independently but exposed as a single device, referred to as a cluster. Each server in the cluster can be implemented as a node.

Client device 780 may be linked to a service application in cloud computing platform 710. Client device 780 may be any type of computing device, such as user device 102n described with reference to FIG. 1, and the client device 780 can be configured to issue commands to cloud computing platform 710. In embodiments, client device 780 may communicate with service applications through a virtual Internet Protocol (IP) and load balancer or other means that direct communication requests to designated endpoints in cloud computing platform 710. The components of cloud computing platform 710 may communicate with each other over a network (not shown), which may include one or more LANs and/or WANs.

### OTHER EMBODIMENTS

In some embodiments, a computing system is provided, employing any components of the computerized (or computer, computing, or cloud) system of any of the embodiments described herein. The computing system comprises at least one computer processor, and computer memory having computer-readable instructions embodied thereon, that, when executed by the at least one computer processor, perform operations. The operations comprise receiving patient data for a human patient, the data including a location on the patient for utilizing an implant. The operations further comprise receiving implant characterization data comprising osteoinduction and osteoconduction data characterizing at least one implant type. The operations further comprise determining one or more applicable implants for the patient by applying the patient data and the implant characterization data to an artificial intelligence (AI) model. The operations further comprise generating a recommendation based on the one or more applicable implants. The operations further comprise causing a representation of the recommendation to be presented via a user interface.

In any combination of the above embodiments of the system, the AI model comprises a machine learning model, a predictive analytical model, or a generative AI model.

In any combination of the above embodiments of the system, the patient data further includes an osteogenic capacity score for the patient.

In any combination of the above embodiments of the system, the AI model is trained to output indications of implants that are optimized with regards to osteoconduction, osteoinduction, or osteogenesis of bone graft material of the implants.

In any combination of the above embodiments of the system, the implants of the AI model training are based on prior health data of patient populations identified as optimal clinical outcomes.

In any combination of the above embodiments of the system, the operations further comprise receiving, via the user interface, a selection indicating a first implant from among the one or more applicable implants, wherein the first implant is a biologic, and in response to receiving the selection, causing the biologic to be prepared via bone graft preparation hardware.

In any combination of the above embodiments of the system, the operations further comprise causing the biologic to be loaded automatically into a robotic system.

In any combination of the above embodiments of the system, the operations further comprise receiving, via the user interface, a selection indicating a first implant from among the one or more applicable implants; and in response to receiving the selection, performing an action comprising at least one of: (a) generating computer instructions corresponding to a care plan for an implant procedure involving the first implant and the patient; and (b) causing display of an aspect of the patient data that is relevant to the first implant, causing display of information regarding an implant procedure corresponding to the first implant, and causing display, via a second user interface associated with the patient, information regarding provisions for the implant from a medical provider that designated to perform an implant procedure on the patient; and (c) determining, using at least a portion of the patient data, a three-dimensional (3D) image including a representation of the first implant and a representation of the patient, and causing the 3D image to be presented via the user interface.

In any combination of the above embodiments of the system, the operations further comprise receiving context data: wherein the generating the recommendation further based on the context data; wherein the patient data comprises surgical history for the patient; and wherein the context data comprises at least one of: preference of a caregiver associated with an implant procedure for the patient, experience of the caregiver associated with the implant procedure for the patient, historical outcomes data regarding prior implant procedures for patients at a medical provider associated with the patient, surgical history for the medical provider associated with the patient, available healthcare resources regarding implants for the medical provider associated with the patient, an administrative criterion, an insurance criterion, a likelihood or readmission, a regulatory requirement, and a procedural protocol that correlates an implant with a biologic.

In any combination of the above embodiments of the system, the generating the recommendation comprises determining a three-dimensional (3D) image for at least a first applicable implant of the one or more applicable implants, and wherein the representation of the recommendation includes at least a portion of a 3D image for a first applicable implant.

In any combination of the above embodiments of the system, the 3D image for the first applicable implant is determined using a digital twin for the patient.

In any combination of the above embodiments of the system, generating the recommendation comprises determining a projected clinical outcome for a first applicable implant of the one or more applicable implants, and wherein the representation of the recommendation includes an indication of the projected clinical outcome.

In any combination of the above embodiments of the system, at least one applicable implant of the one or more applicable implants comprises a biologic, bone graft material, hardware component interbody, or a combination of one or more of these.

In any combination of the above embodiments of the system, at least one applicable implant of the one or more applicable implants comprises a biologic, and wherein the implant characterization data comprises biologic data including one or more of: biochemical properties, surface chemistry, efficacy of one or more bone graft materials, correlation data of bone graft material as utilized with a second implant, osteoconduction, osteoinduction, osteogenesis, physical attributes, scaffold structure, crystalline composition, compression resistance, malleability, hydration capacity, hydration component data, hydration state, dehydration status, rehydration capability, and nanostructural component data.

In any combination of the above embodiments of the system, the patient data further comprises osteogenic data regarding the patient.

In any combination of the above embodiments of the system, the patient data further comprises a biomarker.

In any combination of the above embodiments of the system, the biomarker includes a spinal bone health indicator comprising a DEXA score, ALP, OC, P1NP, P1CP, HYP, DPD, PYD, NTX-1, CTX-1, BSP, TRAP5b, COL1, COL9, COL11, CILP, ASPN, or GDF5.

In any combination of the above embodiments of the system, the operations further comprise receiving, via the user interface, a constraint regarding at least one applicable implant or the one or more applicable implants, the constraint corresponding to availability of bone graft material or preference of a caregiver associated with an implant procedure for the patient, and wherein the representation of the recommendation includes a comparison of a first recommendation generated without regards to the constraint and a second recommendation generated based on the constraint.

In some embodiments, a computer-implemented method is provided. The method comprises receiving patient data for a human patient, the data including a location on the patient for utilizing a spinal implant and an osteogenic capacity score for the patient. The method further comprises receiving implant characterization data comprising osteoinduction and osteoconduction data characterizing at least one spinal implant type. The method further comprises determining one or more applicable implants for the patient by applying at least the patient data and the implant characterization data to an artificial intelligence (AI) model, the AI model trained to output indications of implants that are optimized with regards to osteoconduction, osteoinduction, or osteogenesis of bone graft material of the implants based on prior health data of a patient population identified as optimal clinical outcome. The method further comprises generating a recommendation based on the one or more applicable implants. The method further comprises causing a representation of the recommendation to be presented via a user interface.

In any combination of the above embodiments of the method, generating the recommendation comprises determining a first procedural protocol corresponding to a first applicable implant of the one or more implants.

In any combination of the above embodiments of the method, the representation of the recommendation includes an indication of the first procedural protocol.

In any combination of the above embodiments of the method, the implant characterization data comprises data of other patients in correlation with fusion and pain outcomes associated with interbody cages or biologics.

In any combination of the above embodiments of the method further comprises receiving context data comprising at least one of: preference of a caregiver associated with an implant procedure for the patient, experience of the caregiver associated with the implant procedure for the patient, historical outcomes data regarding prior implant procedures for patients at a medical provider associated with the patient, surgical history for the medical provider associated with the patient, available healthcare resources regarding implants for the medical provider associated with the patient, an administrative criterion, an insurance criterion, a likelihood or readmission, a regulatory requirement, and a procedural protocol that correlates an implant with a biologic.

In any combination of the above embodiments of the method, the one or more applicable implants for the patient are further determined by applying the context data to the AI model.

In some embodiments, a surgical guidance system is provided, employing any components of the computerized (or computer, computing, or cloud) system of any of the embodiments described herein. The surgical guidance system is operative to receive, at a computer processor, feedback data, provided by one or more distributed networked computers, the feedback data for a plurality of prior patients who have undergone spinal surgery and characterizing implant-related data for each prior patient. The surgical guidance system is further operative to train an artificial intelligence (AI) model based on the feedback data. The surgical guidance system is further operative to obtain, from at least one of the one or more distributed network computers, pre-operative data characterizing implants of a first patient for surgery. The surgical guidance system is further operative to generate a surgical plan for the first patient based on processing the pre-operative data through the AI model, the surgical implant regarding an applicable implant for the patient. The surgical guidance system is further operative to cause at least a portion of the surgical plan to be presented on a display device.

In any combination of the above embodiments of the surgical guidance system, the AI model comprises a machine learning model, a predictive analytical model, or a generative AI model.

In any combination of the above embodiments of the surgical guidance system, the feedback data comprises one or more of: data regarding a first implant; a characteristic comprising a structural or biochemical property, or surface chemistry; efficacy of a bone graft material; correlation data of bone graft material as utilized with a second implant; osteoconduction; osteoinduction; osteogenesis; physical attributes; scaffold structure; crystalline composition; compression resistance; malleability; hydration capacity; hydration component; hydration state; dehydration status; rehydration capability; nanostructural components, and including osteogenic capacity scores of a patient.

In any combination of the above embodiments of the surgical guidance system, processing the pre-operative data through the AI model produces a model output that includes the applicable implant.

### Additional Structural and Functional Features of Embodiments of the Technical Solutions

Having identified various components utilized herein, it should be understood that any number of components and arrangements may be employed to achieve the desired functionality within the scope of the present disclosure. For example, the components in the embodiments depicted in the figures are shown with lines for the sake of conceptual clarity. Other arrangements of these and other components may also be implemented. For example, although some components are depicted as single components, many of the elements described herein may be implemented as discrete or distributed components or in conjunction with other components, and in any suitable combination and location. Some elements may be omitted altogether. Moreover, various functions described herein as being performed by one or more entities may be carried out by hardware, firmware, and/or software, as described below. For instance, various functions may be carried out by a processor executing instructions stored in memory. As such, other arrangements and elements (e.g., machines, interfaces, functions, orders, and groupings of functions) can be used in addition to or instead of those shown.

Embodiments described in the paragraphs below may be combined with one or more of the specifically described alternatives. In particular, an embodiment that is claimed may contain a reference, in the alternative, to more than one other embodiment. The embodiment that is claimed may specify a further limitation of the subject matter claimed.

The subject matter of aspects of the present disclosure is described with specificity herein to meet statutory requirements. However, the description itself is not intended to limit the scope of this disclosure. Rather, it is contemplated that the claimed subject matter might also be embodied in other ways, such as to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described. Each method described herein may comprise a computing process that may be performed using any combination of hardware, firmware, and/or software. For instance, various functions may be carried out by a processor executing instructions stored in memory. The methods may also be embodied as computer-useable instructions stored on computer storage media. The methods may be provided by a stand-alone application, a service or hosted service (stand-alone or in combination with another hosted service), or a plug-in to another product, to name a few.

For purposes of this disclosure, the word “including” has the same broad meaning as the word “comprising,” and the word “accessing” comprises “receiving,” “referencing,” or “retrieving.” Furthermore, the word “communicating” has the same broad meaning as the word “receiving,” or “transmitting” facilitated by software or hardware-based buses, receivers, or transmitters using communication media described herein. In addition, words such as “a” and “an,” unless otherwise indicated to the contrary, include the plural as well as the singular. Thus, for example, the constraint of “a feature” is satisfied where one or more features are present. Also, the term “or” includes the conjunctive, the disjunctive, and both; (a or b thus includes either a or b, as well as a and b).

As used herein, the term “set” may be employed to refer to an ordered (i.e., sequential) or an unordered (i.e., non-sequential) collection of objects (or elements), such as machines (e.g., computer devices), physical and/or logical addresses, graph nodes, graph edges, functionalities, and the like. As used herein, a set may include N elements where N is any positive integer. That is, a set may include 1, 2, 3, . . . N objects and/or elements, where N is a positive integer with no upper bound. Therefore, as used herein, a set does not include a null set (i.e., an empty set), that includes no elements (e.g., N=0 for the null set). A set may include only a single element. In other embodiments, a set may include a number of elements that is significantly greater than one, two, three, or billions of elements. A set may be an infinite set or a finite set. The objects included in some sets may be discrete objects (e.g., the set of natural numbers N). The objects included in other sets may be continuous objects (e.g., the set of real numbers R). In some embodiments, “a set of objects” that is not a null set of the objects may be interchangeably referred to as either “one or more objects” or “at least one object,” where the term “object” may stand for any object or element that may be included in a set. Accordingly, the phrases, “one or more objects” and “at least one object” may be employed interchangeably to refer to a set of objects that is not the null or empty set of objects. A set of objects that includes at least two of the objects may be referred to as “a plurality of objects.”

As used herein, the terms “source code” and “code” may be used interchangeably to refer human-readable instructions that at least partially enable the execution of a computer application. Source code maybe encoded in one or more programming languages, e.g., Fortran, C, C++, Python, Ruby, Julia, R, Octave, Java, JavaScript, C#, PERL, Rust, HTML, Prolog, Lisp, Smalltalk, Haskell, and the like. In some embodiments, prior to enabling an execution of a computer application, source code may be subjected to a compilation and/or linking process. As used herein, the term “executable” may refer to any set of machine instructions that instantiate a copy of a computer application and enable the one or more computing machines (e.g., a physical or virtual machine) to execute, run, or otherwise implement the instantiated application. A computer application may include a set of executables. An executable may be a binary executable, e.g., a set of executable machine instructions generated via the compilation of human-readable source code (in a programming language) and linking of the binary objects generated via the compilation. That is, an executable for a computer application may be generated via compiling the source code for the computer application. Although the embodiments are not so limited, a computer application may include human-readable source code, e.g., applications generated via interpreted programming languages. For instance, an executable for the computer application may include the source code for the computer application. An executable may include one or more binary executables, one or more source code-based executables, or any combination thereof. An executable may include and be dependent upon one or more libraries of functions, objects, or the like. An executable may be encoded in a single file, or the encoding may be distributed across multiple files. That is, an encoding of an executable may be distributed across a plurality of files. The encoding may include one or more data files, where the execution of the computer application may be dependent upon reading and/or writing to the one or more data files.

For purposes of a detailed discussion above, embodiments of the present disclosure are described with reference to a computing device or a distributed computing environment; however the computing device and distributed computing environment depicted herein is merely illustrative. Moreover, the terms computer system and computing system may be used interchangeably herein, such that a computer system is not limited to a single computing device, nor does a computing system require a plurality of computing devices. Rather, various aspects of the embodiments of this disclosure may be carried out on a single computing device or a plurality of computing devices, as described herein. Additionally, components can be configured for performing novel aspects of embodiments, where the term “configured for” can refer to “programmed to” perform particular tasks or implement particular abstract data types using code. Further, while embodiments of the present disclosure may generally refer to the technical solution environment and the schematics described herein, it is understood that the techniques described may be extended to other implementation contexts.

Many different arrangements of the various components depicted, as well as components not shown, are possible without departing from the scope of the claims below. Embodiments of the present disclosure have been described with the intent to be illustrative rather than restrictive. Alternative embodiments will become apparent to readers of this disclosure after and because of reading it. Alternative means of implementing the aforementioned can be completed without departing from the scope of the claims below. Certain features and sub-combinations are of utility and may be employed without reference to other features and sub-combinations and are contemplated within the scope of the claims.

1. A computing system comprising:
at least one processor; and
computer memory having computer-readable instructions embodied thereon, that, when executed by the at least one processor, perform operations comprising:
receiving patient data for a human patient, the data including a location on the patient for utilizing an implant;
receiving implant characterization data comprising osteoinduction and osteoconduction data characterizing at least one implant type;
determining one or more applicable implants for the patient by applying the patient data and the implant characterization data to an artificial intelligence (AI) model;
generating a recommendation based on the one or more applicable implants; and
causing a representation of the recommendation to be presented via a user interface.

2. The computing system of claim 1:
wherein the AI model comprises a machine learning model, a predictive analytical model, or a generative AI model;
wherein the patient data further includes an osteogenic capacity score for the patient; and
wherein the AI model is trained to output indications of implants that are optimized with regards to osteoconduction, osteoinduction, or osteogenesis of bone graft material of the implants.

3. The computing system of claim 1, further comprising:
receiving, via the user interface, a selection indicating a first implant from among the one or more applicable implants, wherein the first implant is a biologic; and
in response to receiving the selection, causing the biologic to be prepared via bone graft preparation hardware.

4. The computing system of claim 3, further comprising causing the biologic to be loaded automatically into a robotic system.

5. The computing system of claim 1, further comprising:
receiving, via the user interface, a selection indicating a first implant from among the one or more applicable implants; and
in response to receiving the selection, performing an action comprising at least one of:
(a) generating computer instructions corresponding to a care plan for an implant procedure involving the first implant and the patient;
(b) causing display of an aspect of the patient data that is relevant to the first implant, causing display of information regarding an implant procedure corresponding to the first implant, and causing display, via a second user interface associated with the patient, information regarding provisions for the implant from a medical provider that designated to perform an implant procedure on the patient; and
(c) determining, using at least a portion of the patient data, a three-dimensional (3D) image including a representation of the first implant and a representation of the patient, and causing the 3D image to be presented via the user interface.

6. The computing system of claim 1 further comprising receiving context data:
wherein the generating the recommendation further based on the context data;
wherein the patient data comprises surgical history for the patient; and
wherein the context data comprises at least one of: preference of a caregiver associated with an implant procedure for the patient, experience of the caregiver associated with the implant procedure for the patient, historical outcomes data regarding prior implant procedures for patients at a medical provider associated with the patient, surgical history for the medical provider associated with the patient, available healthcare resources regarding implants for the medical provider associated with the patient, an administrative criterion, an insurance criterion, a likelihood or readmission, a regulatory requirement, and a procedural protocol that correlates an implant with a biologic.

7. The computing system of claim 1, wherein the generating the recommendation comprises determining a three-dimensional (3D) image for at least a first applicable implant of the one or more applicable implants, and wherein the representation of the recommendation includes at least a portion of a 3D image for a first applicable implant.

8. The computing system of claim 7, wherein the 3D image for the first applicable implant is determined using a digital twin for the patient.

9. The computing system of claim 1, wherein generating the recommendation comprises determining a projected clinical outcome for a first applicable implant of the one or more applicable implants, and wherein the representation of the recommendation includes an indication of the projected clinical outcome.

10. The computing system of claim 1, wherein at least one applicable implant of the one or more applicable implants comprises a biologic, bone graft material, hardware component interbody, or a combination of one or more of these.

11. The computing system of claim 1, wherein at least one applicable implant of the one or more applicable implants comprises a biologic, and wherein the implant characterization data comprises biologic data including one or more of: biochemical properties, surface chemistry, efficacy of one or more bone graft materials, correlation data of bone graft material as utilized with a second implant, osteoconduction, osteoinduction, osteogenesis, physical attributes, scaffold structure, crystalline composition, compression resistance, malleability, hydration capacity, hydration component data, hydration state, dehydration status, rehydration capability, and nanostructural component data.

12. The computing system of claim 1, wherein the patient data further comprises osteogenic data regarding the patient.

13. The computing system of claim 1, wherein the patient data further comprises a biomarker.

14. The computing system of claim 13, wherein the biomarker includes a spinal bone health indicator comprising a DEXA score, ALP, OC, P1NP, P1CP, HYP, DPD, PYD, NTX-1, CTX-1, BSP, TRAP5b, COL1, COL9, COL11, CILP, ASPN, or GDF5.

15. The computing system of claim 1, further comprising receiving, via the user interface, a constraint regarding at least one applicable implant or the one or more applicable implants, the constraint corresponding to availability of bone graft material or preference of a caregiver associated with an implant procedure for the patient, and wherein the representation of the recommendation includes a comparison of a first recommendation generated without regards to the constraint and a second recommendation generated based on the constraint.

16. A computer implemented method comprising:
receiving patient data for a human patient, the data including a location on the patient for utilizing a spinal implant and an osteogenic capacity score for the patient;
receiving implant characterization data comprising osteoinduction and osteoconduction data characterizing at least one spinal implant type;
determining one or more applicable implants for the patient by applying at least the patient data and the implant characterization data to an artificial intelligence (AI) model, the AI model trained to output indications of implants that are optimized with regards to osteoconduction, osteoinduction, or osteogenesis of bone graft material of the implants based on prior health data of a patient population identified as optimal clinical outcome;
generating a recommendation based on the one or more applicable implants; and
causing a representation of the recommendation to be presented via a user interface.

17. The computer implemented method of claim 16:
wherein generating the recommendation comprises determining a first procedural protocol corresponding to a first applicable implant of the one or more implants;
wherein the representation of the recommendation includes an indication of the first procedural protocol; and
wherein the implant characterization data comprises data of other patients in correlation with fusion and pain outcomes associated with interbody cages or biologics.

18. The computer implemented method of claim 16, further comprising:
receiving context data comprising at least one of: preference of a caregiver associated with an implant procedure for the patient, experience of the caregiver associated with the implant procedure for the patient, historical outcomes data regarding prior implant procedures for patients at a medical provider associated with the patient, surgical history for the medical provider associated with the patient, available healthcare resources regarding implants for the medical provider associated with the patient, an administrative criterion, an insurance criterion, a likelihood or readmission, a regulatory requirement, and a procedural protocol that correlates an implant with a biologic;
wherein the one or more applicable implants for the patient are further determined by applying the context data to the AI model.

19. A surgical guidance system operative to:
receive, at a computer processor, feedback data, provided by one or more distributed networked computers, the feedback data for a plurality of prior patients who have undergone spinal surgery and characterizing implant-related data for each prior patient;
train an artificial intelligence (AI) model based on the feedback data;
obtain, from at least one of the one or more distributed network computers, pre-operative data characterizing implants of a first patient for surgery;
generate a surgical plan for the first patient based on processing the pre-operative data through the AI model, the surgical implant regarding an applicable implant for the patient; and
cause at least a portion of the surgical plan to be presented on a display device.

20. The surgical guidance system of claim 19:
wherein the AI model comprises a machine learning model, a predictive analytical model, or a generative AI model;
wherein the feedback data comprises one or more of: data regarding a first implant; a characteristic comprising a structural or biochemical property, or surface chemistry; efficacy of a bone graft material; correlation data of bone graft material as utilized with a second implant; osteoconduction; osteoinduction; osteogenesis; physical attributes; scaffold structure; crystalline composition; compression resistance; malleability; hydration capacity; hydration component; hydration state; dehydration status; rehydration capability; nanostructural components, and including osteogenic capacity scores of a patient; and
wherein processing the pre-operative data through the AI model produces a model output that includes the applicable implant.
